(19) United States
(12) Patent Application Publication
(10) Pub. No.: US 2026/0366069 A1
(43) Pub. Date: Nov. 19, 2026

(54) Dual Indexing Specific Binding Members for Obtaining Linked Single Cell Cytometric and Sequence Data

(71) Applicant: Becton, Dickinson and Company, Franklin Lakes, NJ (US)

(72) Inventors: Jody Martin, Ashland, OR (US); Geoffrey Osborne, Bellbowrie (AU); Hye Won Song, San Diego, CA (US); Adam Thomas Wright, La Jolla, CA (US); Cynthia Jean Sakofsky, Fremont, CA (US)

(21) Appl. No.: 19/153,534

(22) PCT Filed: Feb. 26, 2024
(86) PCT No.: PCT/US2024/017296

§ 371 Date: Aug. 4, 2025

Related U.S. Application Data
(60) Provisional application No. 63/448,935, filed on Feb. 28, 2023.

(51) Int. Cl. C12Q 1/6869; C12Q 1/6813; C12Q 1/6876

(52) U.S. Cl. CPC C12Q 1/6869; C12Q 1/6813; C12Q 1/6876

(57) ABSTRACT

Methods and compositions of preparing an indexed population of cells, e.g., which may be used in protocols to obtain linked single cell flow cytometric and sequence (such as multiomic) data, are provided. Aspects of the methods include: splitting a cellular sample into a first plurality of portions; combining different portions of the first plurality with distinct first dual-indexed specific binding members having distinct fluorescence and oligonucleotide barcodes to stably associate cells of the portions with first dual-indexed specific binding members; combining portions of the first plurality to produce a first pool; splitting the first pool into a second plurality of portions; and combining different portions of the second plurality with distinct second dual-indexed specific binding members having distinct fluorescence and oligonucleotide barcodes to stably associate cells of the portions with second dual-indexed specific binding members; to produce an indexed population of cells. In embodiments, the resultant indexed population of cells is then subjected to flow cytometric and sequence workflows, where the obtained flow cytometric data and sequencing data may be linked. Also provided are compositions for use in practicing the methods.

### CROSS REFERENCE TO RELATED APPLICATION

This application claims priority to the filing date of U.S. Provisional Patent Application Ser. No. 63/448,935 filed on Feb. 28, 2023; the disclosure of which application is incorporated herein by reference.

### INTRODUCTION

Current technology allows for measurement of gene expression of single cells in a massively parallel manner (e.g., >10,000 cells) by attaching cell specific oligonucleotide barcodes to poly(A) mRNA molecules from individual cells as each of the cells is co-localized with a barcoded reagent bead in a compartment. One platform that allows measurement of gene expression of single cells in a massively parallel manner is the BD Rhapsody™ Single-Cell Analysis System. The BD Rhapsody™ Single-Cell Analysis System is a platform that allows high-throughput capture of nucleic acids from single cells using a simple cartridge workflow and a multitier barcoding system. The resulting captured material can be used to generate various types of next-generation sequencing (NGS) libraries, including libraries suitable for whole transcriptome analysis, e.g., for discovery biology and targeted RNA analysis for high sensitivity transcript detection. Shum et al., “Quantitation of mRNA Transcripts and Proteins Using the BD Rhapsody™ Single-Cell Analysis System,” Adv Exp Med Biol. 2019; 1129:63-79.

Gene expression may affect protein expression. Protein-protein interaction may affect gene expression and protein expression. As such, more recently systems and methods that can quantitatively analyze protein expression in cells, and simultaneously measure protein expression and gene expression in cells, have been developed. One such platform is the BD Abseq platform. AbSeq is a method to profile proteins in and on single cells. In Abseq, the usual fluorophore labels on antibodies are replaced with nucleic acid sequence tags that can be read out at the single-cell level, e.g., via barcoding and NGS sequencing. “The objective of Abseq is to enable the sensitive, accurate, and comprehensive characterization of proteins along with mRNA Transcripts in large numbers of single cells. Cells are bound with antibodies against the different target epitopes, as in conventional immunostaining, except that the antibodies are labeled with unique sequence tags. When an antibody binds its target, the DNA tag is carried with it, allowing the presence of the target to be inferred based on the presence of the tag. In this way, counting tags provides an estimate of the different epitopes present in the cell, as detected via antibody binding.” Shahi et al., “Abseq: Ultrahigh-throughput single cell protein profiling with droplet microfluidic barcoding. Sci Rep 7, 44447 (2017).”

Flow cytometry is a technique used to characterize, e.g., detect, measure, etc., physical and/or chemical characteristics of a cellular sample. In flow cytometry, a cellular sample is suspended in a fluid and injected into the flow cytometer instrument. The sample is focused to ideally flow one cell at a time through a laser beam, where the light scattered is characteristic to the cells and their components. Cells are often labeled with fluorescent markers (e.g., antibody-fluorophore) so light is absorbed and then emitted in a band of wavelengths. Tens of thousands of cells can be quickly examined, and data gathered therefrom. Flow cytometry is routinely used in basic research, clinical practice, and clinical trials. Applications in which flow cytometry is employed include, but are not limited to: cell counting; cell sorting; determining cell characteristics and function; detecting microorganisms; biomarker detection; protein engineering detection; Diagnosis of health disorders; measuring genome size; etc. A flow cytometry analyzer is an instrument that provides quantifiable data from a sample. Other instruments using flow cytometry include cell sorters which physically separate and thereby purify cells of interest based on their optical properties.

### SUMMARY

The inventors have realized that it would be desirable to be able to link flow cytometry data with downstream sequence data, e.g., multiomic data, such that flow cytometry, including image cytometry data and sequence, data can be readily obtained for the same cell. Described here is a novel method for indexing individual cells for correlation of fluorescence data (immunophenotyping, functional, or other) or brightfield imaging data to downstream single cell sequence, e.g., multiomics, data. Embodiments of the invention provide for the ability to select live or preserved cells of interest via imaging and/or fluorescence analysis (such as Fluorescence Activated Cell Sorting) and then obtain sequence data for such selected cells.

Methods and compositions of preparing an indexed population of cells, e.g., which may be used in protocols to obtain linked single cell flow cytometric and sequence (such as multiomic) data, are provided. Aspects of the methods include: splitting the cellular sample into a first plurality of portions; combining different portions of the first plurality with distinct first dual-indexed specific binding members having distinct fluorescence and oligonucleotide barcodes to stably associate cells of the portions with first dual-indexed specific binding members; combining portions of the first plurality to produce a first pool; splitting the first pool into a second plurality of portions; and combining different portions of the second plurality with distinct second dual-indexed specific binding members having distinct fluorescence and oligonucleotide barcodes to stably associate cells of the portions with second dual-indexed specific binding members; to produce an indexed population of cells. Individual cell indexing is a result of unique combinatorial labeling of the cell. In embodiments, the resultant indexed population of cells is then subjected to flow cytometric and sequence workflows, where the obtained flow cytometric data and sequencing data may be linked. Also provided are compositions for use in practicing the methods.

Aspects of the invention utilize dual-indexed specific binding members, having associated oligo barcodes as well as a fluorescence address (i.e., fluorescence barcode). Aspects of the methods employ a large and diverse collection of dual-indexed specific binding members. Within the collection, a unique oligo barcode is directly associated with each unique fluorescence barcode. In embodiments, the dual-indexed specific binding members are randomly associated with cells, e.g., via a combinatorial protocol, such as a split/pool protocol. Association of a random combination of dual-indexed specific binding members with individual cells provides a unique combination of barcodes associated with cells in the sample, such that the majority of, if not all of, the cells have a unique fluorescence signature made up of the one or more barcodes of the dual indexed specific binding members associated with that cell. The fluorescence barcode(s) of cell-associated specific binding members may then be analyzed utilizing the same modality collecting fluorescence data from the cell (such as flow cytometry). Subsequently, the oligo barcodes of the dual-indexed specific binding members may be released and captured by complementary oligos in a single cell multiomics workflow, at the same time as endogenous nucleic acid and other barcodes associated with cells, e.g., antibody associated barcodes, are captured upon cell lysis. The oligo barcodes from dual-indexed specific binding members may be analogous to Sample Multiplexing oligos in routine cell Hashing experiments. Standard single cell multiomic library preparation, sequencing protocols, and data analysis may then be used to identify the combination of oligo barcodes unique to every cell in the experiment. With direct association between every combo-oligo barcode and every combo-fluorescence barcode, the NGS single cell data can be one to one correlated with the flow cytometry single cell data.

### BRIEF DESCRIPTION OF THE FIGURES

The invention may be best understood from the following detailed description when read in conjunction with the accompanying drawings. Included in the drawings are the following figures:

FIG. 1 provides a schematic of a workflow in accordance with an embodiment of the invention.

FIG. 2 provides a schematic of a workflow in accordance with another embodiment of the invention.

FIG. 3 provides details regarding dual indexed reagents that may be employed in embodiments of the invention.

### DEFINITIONS

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, an antibody can be a full-length (e.g., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. In some embodiments, an antibody is a functional antibody fragment. For example, an antibody fragment can be a portion of an antibody such as F(ab′)2, Fab′, Fab, Fv, sFv and the like. An antibody fragment can bind with the same antigen that is recognized by the full-length antibody. An antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (for example, CD8, CD34, and CD45), and therapeutic antibodies.

As used herein the term “associated” or “associated with” can mean that two or more species are identifiable as being co-located at a point in time. An association can mean that two or more species are or were within a similar container. An association can be an informatics association. For example, digital information regarding two or more species can be stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label. An association can comprise hybridization between two molecules (such as a target molecule and a label).

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (e.g., morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tm=+ 3 to +10 °C), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g., adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one).

As used herein, the term “sample” can refer to a composition comprising targets. Suitable samples for analysis by the disclosed methods, devices, and systems include cells, tissues, organs, or organisms. A cellular sample is a composition that is made up of multiple cells, such as a composition that includes multiple disparate cells, such as an aqueous composition of single cells, where the number of cells may vary.

As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which nucleic acids may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A plurality of solid supports spaced in an array may not comprise a substrate. A solid support may be used interchangeably with the term “bead.”

As used here, the term “target” can refer to a composition which can be analyzed in accordance with embodiments of the invention. Exemplary suitable targets for analysis by the disclosed methods, devices, and systems include oligonucleotides, DNA, RNA, mRNA, microRNA, tRNA, and the like. Targets can be single or double stranded. In some embodiments, targets can be proteins, peptides, or polypeptides. In some embodiments, targets are lipids. As used herein, “target” can be used interchangeably with “species.”

As used herein, the term “reverse transcriptases” can refer to a group of enzymes having reverse transcriptase activity (i.e., that catalyze synthesis of DNA from a RNA template). In general, such enzymes include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants or derivatives thereof. Non-retroviral reverse transcriptases include non-LTR retrotransposon reverse transcriptases, retroplasmid reverse transcriptases, retron reverse transciptases, and group II intron reverse transcriptases. Examples of group II intron reverse transcriptases include the Lactococcus lactis LI.LtrB intron reverse transcriptase, the Thermosynechococcus elongatus Tel4c intron reverse transcriptase, or the Geobacillus stearothermophilus Gsl-IIC intron reverse transcriptase. Other classes of reverse transcriptases can include many classes of non-retroviral reverse transcriptases (i.e., retrons, group II introns, and diversity-generating retroelements among others).

The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25 °C “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25 °C.

The specific binding member can be proteinaceous. As used herein, the term “proteinaceous” refers to a moiety that is composed of amino acid residues. A proteinaceous moiety can be a polypeptide. In certain cases, the proteinaceous specific binding member is an antibody. In certain embodiments, the proteinaceous specific binding member is an antibody fragment, e.g., a binding fragment of an antibody that specifically binds to a a target analyte. As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (k), lambda (I), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. The term antibody is meant to include full length antibodies and may refer to a natural antibody from any organism, an engineered antibody, or an antibody generated recombinantly for experimental, therapeutic, or other purposes as further defined below.

Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions.

In certain embodiments, the specific binding member is a Fab fragment, a F(ab′)2 fragment, a scFv, a diabody or a triabody. In certain embodiments, the specific binding member is an antibody. In some cases, the specific binding member is a murine antibody or binding fragment thereof. In certain instances, the specific binding member is a recombinant antibody or binding fragment thereof.

### DETAILED DESCRIPTION

Methods and compositions of preparing an indexed population of cells, e.g., which may be used in protocols to obtain linked single cell flow cytometric and sequence (such as multiomic) data, are provided. Aspects of the methods include: splitting a cellular sample into a first plurality of portions; combining different portions of the first plurality with distinct first dual-indexed specific binding members having distinct fluorescence and oligonucleotide barcodes to stably associate cells of the portions with first dual-indexed specific binding members; combining portions of the first plurality to produce a first pool; splitting the first pool into a second plurality of portions; and combining different portions of the second plurality with distinct second dual-indexed specific binding members having distinct fluorescence and oligonucleotide barcodes to stably associate cells of the portions with second dual-indexed specific binding members; to produce an indexed population of cells. In embodiments, the resultant indexed population of cells is then subjected to flow cytometric and sequence workflows, where the obtained flow cytometric data and sequencing data may be linked. Also provided are compositions for use in practicing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of preparing an indexed population of cells are provided. In some instances, the indexed population of cells is made up of a plurality of distinguishably indexed cells, where distinguishably indexed cells of the plurality have different or unique fluorescence signatures associated therewith. By “fluorescent signature” (i.e., fluorescent identifier) is meant composite or collective spectrum made up of one or more fluorescent emission signals obtained from one or more fluorophores stably associated with the indexed cell, e.g., provided by dual indexed specific binding members (such as described in greater detail below) associated with the cell. Different cells of the plurality of cells in the indexed population of cells produced by methods of embodiments of the invention have distinguishable fluorophores making up their associated fluorescent identifier associated therewith, and therefore provide different fluorescent signatures, e.g., when assayed by flow cytometric protocols. In this way, different indexed cells of the population are distinguishable from each other by the presence of their unique fluorescent signature.

### Dual-Indexed Specific Binding Members

As indicated above, a fluorescent signature of a given indexed cell produced by embodiments of the invention is provided by dual-indexed specific binding members stably associated with the cell. Dual-indexed specific binding members are specific binding members that include one or more fluorophores, where the one or more fluorophores collectively make up the fluorescence barcode of the dual-indexed specific binding member, as well as an oligonucleotide barcode. Dual-indexed specific binding members may vary as desired, and include a specific binding member component, a fluorescent barcode component and an oligonucleotide barcode component.

### Specific Binding Member

The specific binding member component of the dual-indexed specific binding members may vary, where examples of suitable specific binding members include, but are not limited to, those described above. In some instances, the specific binding member is an antibody or antibody fragment, e.g., a binding fragment of an antibody that specifically binds to a target analyte. As used herein, the terms “antibody” and “antibody molecule” are used interchangeably and refer to a protein consisting of one or more polypeptides substantially encoded by all or part of the recognized immunoglobulin genes. Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions. In certain embodiments, the specific binding member is a Fab fragment, a F(ab′)2 fragment, a scFv, a diabody or a triabody. In certain embodiments, the specific binding member is an antibody. In some cases, the specific binding member is a murine antibody or binding fragment thereof. In certain instances, the specific binding member is a recombinant antibody or binding fragment thereof.

### Fluorescent Barcode

The fluorescent barcode of a given dual-indexed specific binding member includes one or more fluorescent dyes. Where a given fluorescent barcode includes more than one fluorescent dye, the two or more fluorescently dyes collectively make up the fluorescent barcode of the dual indexed bead. As such, a given fluorescent barcode may, in embodiments of the invention, be made up of a single fluorescent dye, or two or more fluorescent dyes, e.g., 2 to 20, such as 2 to 10, including 2 to 5, e.g., 2 to 3, fluorescent dyes, which collectively make up the fluorescent barcode of the bead. As such, the number of different fluorophores making up a given fluorescent barcode may vary, ranging in some instances from 1 to 10, such as 1 to 5 and including 1 to 3. Any given two distinguishable fluorescent barcodes may be distinguishable from each other based on the types of fluorophores and/or signal brightness provided thereby. As such, any two distinguishable fluorescent barcodes may be distinguishable based on fluorescent signals (e.g., emission wavelength maxima) and/or intensity thereof, of the fluorescent dyes and/or amount thereof collectively making up the fluorescent barcode. For example, two distinguishable fluorescent barcodes may be distinguishable from each other because they are made up of combinations of different types fluorophore dyes, e.g., where one includes fluorophores a, b and c and the other includes fluorophores b, c and d. Two distinguishable fluorescent barcodes may also be distinguishable from each other because they are made up of different amounts of fluorescent dyes, e.g., where one is made up of fluorophores a, b and c present in a first amount present in a given dual-indexed specific binding member and the other is made up of fluorophores present at a second amount that differs from the first amount at a value that can be detected, e.g., by a difference in brightness of signal. Combinations of type and amount of fluorophores may be employed to provide any desired number of unique fluorescent barcodes.

Fluorescent barcodes include one or more fluorophores, as desired. As such, a dual-indexed specific binding member may include a single type of fluorophore. Alternatively, a given dual-indexed specific binding member may include two or more different types of fluorophores. Examples of fluorophores include, but are not limited to: acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-amino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; Alexa-Fluor dyes (e.g., Alexa Fluor 350, Alexa Fluor 430, Alexa Fluor 488, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, Alexa Fluor 750), Pacific Blue, Pacific Orange, Cascade Blue, Cascade Yellow; Quantum Dot dyes (Quantum Dot Corporation); Dylight dyes from Pierce (Rockford, IL), including Dylight 800, Dylight 680, Dylight 649, Dylight 633, Dylight 549, Dylight 488, Dylight 405; or combinations thereof. Other fluorophores or combinations thereof known to those skilled in the art may also be used, for example those available from Molecular Probes (Eugene, Oreg.) and Exciton (Dayton, Ohio).

In some instances, the fluorophore is a polymeric dye (e.g., fluorescent polymeric dye). Fluorescent polymeric dyes that find use in the subject methods are varied. In some instances of the method, the polymeric dye includes a conjugated polymer. Conjugated polymers (CPs) are characterized by a delocalized electronic structure which includes a backbone of alternating unsaturated bonds (e.g., double and/or triple bonds) and saturated (e.g., single bonds) bonds, where IT-electrons can move from one bond to the other. As such, the conjugated backbone may impart an extended linear structure on the polymeric dye, with limited bond angles between repeat units of the polymer. For example, proteins and nucleic acids, although also polymeric, in some cases do not form extended-rod structures but rather fold into higher-order three-dimensional shapes. In addition, CPs may form “rigid-rod” polymer backbones and experience a limited twist (e.g., torsion) angle between monomer repeat units along the polymer backbone chain. In some instances, the polymeric dye includes a CP that has a rigid rod structure. The structural characteristics of the polymeric dyes can have an effect on the fluorescence properties of the molecules.

Any convenient polymeric dye may be utilized in the subject devices and methods. In some instances, a polymeric dye is a multichromophore that has a structure capable of harvesting light to amplify the fluorescent output of a fluorophore. In some instances, the polymeric dye is capable of harvesting light and efficiently converting it to emitted light at a longer wavelength. In some cases, the polymeric dye has a light-harvesting multichromophore system that can efficiently transfer energy to nearby luminescent species (e.g., a “signaling chromophore”). Mechanisms for energy transfer include, for example, resonant energy transfer (e.g., Forster (or fluorescence) resonance energy transfer, FRET), quantum charge exchange (Dexter energy transfer), and the like. In some instances, these energy transfer mechanisms are relatively short range; that is, close proximity of the light harvesting multichromophore system to the signaling chromophore provides for efficient energy transfer. Under conditions for efficient energy transfer, amplification of the emission from the signaling chromophore occurs when the number of individual chromophores in the light harvesting multichromophore system is large; that is, the emission from the signaling chromophore is more intense when the incident light (the “excitation light”) is at a wavelength which is absorbed by the light harvesting multichromophore system than when the signaling chromophore is directly excited by the pump light.

The multichromophore may be a conjugated polymer. Conjugated polymers (CPs) are characterized by a delocalized electronic structure and can be used as highly responsive optical reporters for chemical and biological targets. Because the effective conjugation length is substantially shorter than the length of the polymer chain, the backbone contains a large number of conjugated segments in close proximity. Thus, conjugated polymers are efficient for light harvesting and enable optical amplification via Forster energy transfer.

Polymeric dyes of interest include, but are not limited to, those dyes described in U.S. Pat. Nos. 7,270,956; 7,629,448; 8,158,444; 8,227,187; 8,455,613; 8,575,303; 8,802,450; 8,969,509; 9,139,869; 9,371,559; 9,547,008; 10,094,838; 10,302,648; 10,458,989; 10,641,775 and 10,962,546 the disclosures of which are herein incorporated by reference in their entirety; and Gaylord et al., J. Am. Chem. Soc., 2001, 123 (26), pp 6417-6418; Feng et al., Chem. Soc. Rev., 2010, 39, 2411-2419; and Traina et al., J. Am. Chem. Soc., 2011, 133 (32), pp 12600-12607, the disclosures of which are herein incorporated by reference in their entirety. Specific polymeric dyes that may be employed include, but are not limited to, BD Horizon Brilliant™ Dyes, such as BD Horizon Brilliant™ Violet Dyes (e.g., BV421, BV510, BV605, BV650, BV711, BV786); BD Horizon Brilliant™ Ultraviolet Dyes (e.g., BUV395, BUV496, BUV737, BUV805); and BD Horizon Brilliant™ Blue Dyes (e.g., BB515, BB550, BB790) (BD Biosciences, San Jose, CA). Any fluorochromes that are known to a skilled artisan—including, but not limited to, those described above—or are yet to be discovered may be employed in the subject methods.

In some instances, each of the fluorophores that make up a given barcode is excitable by common light source, such as a common laser. In such instances, each of the plurality of fluorophores that make up a given barcode may have a common excitation wavelength range (e.g., they are excited by a range of wavelengths that differ from each other by 50 nm or less, such as 25 nm or less, including 10 nm or less, e.g., 5 nm or less), but differ from each other in terms of emission maximum. In such instances, each of the plurality of fluorophores that make up a given barcode may have a common excitation maximum, but differ from each other in terms of emission maximum.

As reviewed above, any given two distinguishable fluorescent barcodes may be distinguishable from each other based on the types of fluorophores make up the barcode and/or signal brightness provided thereby. As such, any two different barcodes may be distinguishable based on fluorescent signals and/or intensity thereof, of the fluorescent signals obtained from the barcode. For example, two distinguishable fluorescent barcodes may be distinguishable from each other because they are made up of combinations of different types fluorophores, e.g., where one includes fluorophores a, b and c and the other includes fluorophores b, c and d. Two distinguishable fluorescent barcodes may also be distinguishable from each other because they are made up of different amounts of fluorophores, e.g., where one is made up of fluorophores a, b and c present in a first amount associated with the dual indexed bead and the other is made up of fluorophores present at a second amount that differs from the first amount at a value that can be detected, e.g., by a difference in brightness of signal. Different brightnesses may readily be provided by having differing amounts of fluorophores associated with the dual indexed bead. Combinations of type and amount of fluorophores may be employed to provide any desired number of unique fluorescent barcodes.

### Oligonucleotide Barcode

In addition to the fluorescent barcode, dual-indexed specific binding members employed in embodiments of the invention include an oligonucleotide barcode. Oligonucleotide barcodes may vary in length, ranging in some instances from 10 to 500 nt, such as 15 to 100 nt. In some instances, the oligonucleotide barcode may be made up of ribonucleic acids or deoxyribonucleic acids, as desired. Oligonucleotide barcodes of embodiments of the invention may include a dual-indexed specific binding member barcode domain, as well as other domains that find use in embodiments of the invention, where such domains may include, a capture sequence, a primer binding site, etc.

The oligonucleotide barcode may include one or more of a dual-indexed specific binding member barcode domain, a capture sequence, a primer binding site and the like. A dual-indexed specific binding member barcode domain is a unique identifier and is a domain or region that may be employed, e.g., by its sequence, to identify the dual-indexed specific binding member with which it is associated. The unique identifiers can be, for example, a nucleotide sequence having any suitable length, for example, from about 4 nucleotides to about 200 nucleotides. In some embodiments, the unique identifier is a nucleotide sequence of 25 nucleotides to about 45 nucleotides in length. In some embodiments, the unique identifier can have a length that is, is about, is less than, is greater than, 4 nucleotides, 5 nucleotides, 6 nucleotides, 7 nucleotides, 8 nucleotides, 9 nucleotides, 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 70 nucleotides, 80 nucleotides, 90 nucleotides, 100 nucleotides, 200 nucleotides, or a range that is between any two of the above values.

The oligonucleotide barcode may include a capture sequence, e.g., which is a domain or region that serves as a binding site for target binding region, e.g., of a bead bound barcode nucleic acid, such as described above. Capture sequences of interest may vary, as desired, and may be specific or random or semi random. In some instances, the capture sequence is a sequence that hybridizes to a target binding region of a bead bound nucleic acids, e.g., as described in greater detail below. In some instances, the capture sequence is a poly(A) sequence, which poly(A) sequence is configured to hybridize to an oligodT target binding region, such as described in greater detail below. In such instances, the length of the poly(A) capture sequence may vary, ranging in some instances from 3 to 50, such as 5 to 25 nt. When present, the capture sequence may be positioned at the 5′ end of the oligonucleotide component.

Oligonucleotide barcodes of dual-indexed specific binding members may include a primer binding site. A primer binding site, when present, may be configured to bind to a primer employed, e.g., in preparing sequence-able nucleic acids. For example, an oligonucleotide component may include a universal primer. A universal primer can refer to a nucleotide sequence that is universal or common across all specific binding member/oligonucleotide sub-barcodes employed in a given workflow. In some instances, a primer binding site can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these nucleotides in length. A primer binding site can vary in length, and can be at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 nucleotides in length. A universal primer can vary in length, and in some instances can range from 5-30 nucleotides in length. The primer binding site can be positioned at the 5′ end of the oligonucleotide barcode component.

As reviewed in greater detail below, in indexing cells of a cellular sample, a cellular sample is indexed with a plurality of distinct dual-indexed specific binding members using a combinatorial, e.g., split/pool, indexing protocol. Among the plurality of distinct dual-indexed specific binding members employed in a given indexing protocol, the oligonucleotide barcodes may share common domains. For example, the oligonucleotide barcodes of the distinct dual-indexed specific binding members may have common capture domains, primer binding sites, etc. In such instances, the capture domains, primer binding sites, and other common domains may have the same sequences, such that the distinct dual-indexed specific binding members of the plurality have identical common sequences, e.g., identical primer binding sites, identical capture domains, etc.

### Indexing Cells of a Cellular Sample With a Plurality of Dual-Indexed Specific Binding Members

As summarized above, methods of embodiments of the invention provide for a plurality of distinguishably indexed cells that each have a different fluorescent signature associated therewith, where a given fluorescent signature is made up of fluorescent barcode(s) provided by dual-indexed specific binding members associated with the cell. While the number of different dual-indexed specific binding members associated with a given indexed cell may vary, in some instances the number ranges from 1 to 10, such as 1 to 5, including 1 to 4, such as 1 to 3, e.g., 1 to 2, including 1, 2 or 3. Each different dual-indexed specific binding member has its own unique fluorescent barcode, and the collection of distinct fluorescent barcodes of the dual-indexed specific binding members associated with the cell collectively provide for the fluorescent signature associated with the indexed cell.

In practicing embodiments of the methods, a cellular sample comprising a plurality of cells is provided. While the number of cells in a given cellular sample may vary, in some instances the number of cells ranges from 50 to 50,000,000, such as 100 to 1,000,000 and including 500 to 100,000. Cells present in a given cellular sample may be any type of cell, including prokaryotic and eukaryotic cells. Suitable prokaryotic cells include, but are not limited to, bacteria such as E. coli, various Bacillus species, and the extremophile bacteria such as thermophiles, etc. Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tulapia, tuna, carp, flounder, halibut, swordfish, cod and zebrafish. Suitable bird cells include, but are not limited to, those of chickens, ducks, quail, pheasants and turkeys, and other jungle foul or game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes. Suitable cells also include those cell types implicated in a wide variety of disease conditions, even while in a non-diseased state. Accordingly, suitable eukaryotic cell types include, but are not limited to, tumor cells of all types (e.g., melanoma, myeloid leukemia, carcinomas of the lung, breast, ovaries, colon, kidney, prostate, pancreas and testes), cardiomyocytes, dendritic cells, endothelial cells, epithelial cells, lymphocytes (T-cell and B cell), mast cells, eosinophils, vascular intimal cells, macrophages, natural killer cells, erythrocytes, hepatocytes, leukocytes including mononuclear leukocytes, stem cells such as haemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. In certain embodiments, the cells are primary disease state cells, such as primary tumor cells. Suitable cells also include known research cells, including, but not limited to, Jurkat T cells, NIH3T3 cells, CHO, COS, etc. See the ATCC cell line catalog, hereby expressly incorporated by reference.

In certain embodiments, the cells used in the present invention are taken from a subject. As used herein “subject” refers to both human and other animals as well as other organisms, such as experimental animals. Thus, the methods and compositions described herein are applicable to both human and veterinary applications. In certain embodiments the subject is a mammal, including embodiments in which the subject is a human patient either having (or suspected of having) a disease or pathological condition.

In certain embodiments, the cells being analyzed are enriched prior to indexing, e.g., as described in greater detail below. For example, if the cells of interest are white blood cells derived from a human subject, whole blood from the subject may be subjected to density gradient centrifugation to enrich for peripheral blood mononuclear cells (PBMCs, or white blood cells). Cells may be enriched using any convenient method known in the art, including fluorescence activated cell sorting (FACS), magnetically activated cell sorting (MACS), density gradient centrifugation and the like. Parameters employed for enriching certain cells from a mixed population include, but are not limited to, physical parameters (e.g., size, shape, density, etc.), in vitro growth characteristics (e.g., in response to specific nutrients in cell culture), and molecule expression (e.g., expression of cell surface proteins or carbohydrates, reporter molecules, e.g., green fluorescent protein, etc.).

In certain embodiments, the cells are live cells which retain viability during the course of the assay. By “retain viability” is meant that a certain percentage of the cells remain alive at the conclusion of the assay, including from about 20% viable up to and including about 100% viable. In certain other embodiments, the methods of the present invention are carried out in such a manner as the cells are rendered non-viable during the course of the assay, e.g., the cells may be fixed, permeabilized, or otherwise maintained in buffers or under conditions in which the cells do not survive. Such parameters are generally dictated by the nature of the assay being performed as well as the reagents being employed.

In some instances, the cells may be treated, e.g., with a stimulus. Stimuli with which cells may be treated may vary, ranging from culture conditions, exposure to changes in temperature, e.g., heat or cold, exposure to electromagnetic radiation, e.g., light, exposure to active agents, exposure to mechanical changes, etc. As desired, different cellular samples of the plurality may be treated with the same or different stimulus. As such, in some instances the method includes differentially treating two or more of the plurality of cellular samples, e.g., where two or more different sample are contacted with different active agents, or different concentrations of the same active agent, etc.

In practicing embodiments of the invention, methods include indexing cells of a cellular sample with a plurality of distinct dual-indexed specific binding members, e.g., as described above, using a combinatorial protocol. By combinatorial protocol is meant a protocol in which cells are indexed by random combinations of different dual-indexed specific binding members selected from population or collection of dual-indexed specific binding members. In some instances, the combinatorial protocol is a split/pool protocol. In some instances, a split/pool protocol employed in embodiments of the invention is one in which an initial cellular sample is subjected to two or more iterations of apportionment into a plurality of portions, contacting different portions of the plurality, such as each portion of the plurality, with a different distinct dual-indexed specific binding member, and then pooling the different dual-indexed specific binding member contacted portions. The number of iterations of split/contact/pool employed in a given split/pool protocol may vary, where in some instances the number of iterations ranges from two to five, such as two to four, e.g., two to three.

In some instances, a given split/pool protocol for producing indexed cells from an initial cellular sample includes first splitting the cellular sample into a first plurality of portions, i.e., aliquots or volumes. The number of different portions in the first plurality may vary, as desired, where in some instances the number ranges from 2 to 25,000 portions, such as 10 to 10,000 portions, and including 20 to 1,000 portions, e.g., 50 to 500 portions, for example, 96 to 384 portions. Each portion is made up of, i.e., includes, a plurality of cells. While the number of cells making up a given sub-portion of the first plurality may vary, in some instances the number ranges from 1 to 10,000, such as 10 to 1,000 and including from 100 to 1,000.

Portions employed in methods of the invention may take a variety of different formats. Portions may take the form of any suitable reaction vessel, including but not limited to e.g., tubes, wells of a multi-well plate, etc. In some instances, portions are wells of a multi-well device (such as e.g., a multi-well plate or a multi-well chip or droplets or the like). Reaction vessels that may serve as portions into which the reaction mixtures and components thereof may be added and within which the reactions of the subject methods may take place will vary. Useful reaction vessels include but are not limited to e.g., tubes (e.g., single tubes, multi-tube strips, etc.), wells (e.g., of a multi-well plate (e.g., a 96-well plate, 384 well plate, or a plate with any number of wells such as 2000, 4000, 6000, or 10,000 or more). Multi-well plates may be independent or may be part of a chip and/or device, e.g., as described in greater detail below. For example, a 96-well plate, 384 well plate, or a plate with any number of wells such as 2000, 4000, 6000, or 10000 or more. The multi-well plate can be part of a chip and/or device. The present disclosure is not limited by the number of wells in the multi-well plate. In various embodiments, the total number of wells on the plate is from 100 to 200,000, or from 5,000 to 10,000. In other embodiments the plate comprises smaller chips, each of which includes 5,000 to 20,000 wells. For example, a square chip may include 125 by 125 nanowells, with a diameter of 0.1 mm. The wells (e.g., nanowells) in the multi-well plates may be fabricated in any convenient size, shape or volume. The wells may be 100 μm to 1 mm in length, 100 μm to 1 mm in width, and 100 μm to 5 mm, or more in depth. In some instances, the wells may have a depth of 5 mm or less, including but not limited to e.g., 4 mm or less, 3 mm or less, 2 mm or less, 1 mm or less. In various embodiments, each nanowell has an aspect ratio (ratio of depth to width) of from 1 to 6 or more. In one embodiment, each nanowell has an aspect ratio of 1:6. The transverse sectional area may be circular, elliptical, oval, conical, rectangular, triangular, polyhedral, or in any other shape. The transverse area at any given depth of the well may also vary in size and shape. In certain embodiments, the wells have a volume of from 0.1 nL to 1 mL. The nanowell may have a volume of 1 μL or less, such as 500 nL or less. The volume may be 200 nL or less, such as 100 nL or less. In an embodiment, the volume of the nanowell is 100 nL. Where desired, the nanowell can be fabricated to increase the surface area to volume ratio, thereby facilitating heat transfer through the unit, which can reduce the ramp time of a thermal cycle. The cavity of each well (e.g., nanowell) may take a variety of configurations. For instance, the cavity within a well may be divided by linear or curved walls to form separate but adjacent compartments, or by circular walls to form inner and outer annular compartments.

Following splitting of the cellular sample into a first plurality of portions, methods of embodiments of the invention include combining different portions of the first plurality with distinct first dual-indexed specific binding members having distinct fluorescence and oligonucleotide barcodes to stably associate cells of the portions with first dual-indexed specific binding members, e.g., as described above. In this step, portions of the plurality are contacted with a dual-indexed specific binding member, where the dual-indexed specific binding member with a given portion is distinct from the dual-indexed specific binding member contacted with another portion, such that each portion that is contacted with the dual-indexed specific binding member is contacted with its own unique dual-indexed specific binding member. Contact of a portion with a dual-indexed specific binding member results in stable association of the dual-indexed specific binding member with cellular constituents of the portion. In embodiments the portions and dual-indexed specific binding members are combined in a liquid, e.g., aqueous, composition. Combination may be achieved under any suitable conditions that provide for stable association of dual-indexed specific binding members with cells of the cellular composition. The dual-indexed specific binding members may be contacted with cells of the cellular sample, e.g., by introducing the dual-indexed specific binding members into a container of the portion (e.g., reaction vessel, such as well), such as by manual or automated fluid dispensing. The portion and dual-indexed specific binding members are combined in a manner such that dual-indexed specific binding members become stably associated with cells of the cellular sample, resulting in indexed cells. The cells and dual-indexed specific binding members may be combined with mixing, as desired, and incubated for a period of time at a temperature suitable to provide for stable association of the dual-indexed specific binding members with the cells. In some instances, the combination of cells and dual-indexed specific binding members is incubated for a period of time ranging from 15 to 120 min, such as 30 to 90 min (e.g., 60 min), at a temperature ranging from 20 to 25 °C, such as 20 to 22 °C.

Next, portions of the first plurality are combined with each other to produce a first pool. Following production of first dual-indexed specific binding member contacted portions, the resultant portions are combined or pooled to produce a first pool of cells comprising first dual-indexed specific binding members. The cells of the different portions may be combined or pooled using any convenient protocol. The number of cells in the resultant first pool may vary, and in some instances ranges from 2 to 10,000,000 cells, such as 10,000 to 1,000,000 cells or 10,000 to 100,000 cells.

Methods of embodiments of the invention then include splitting the first pool into a second plurality of portions. As such, following pooling, the resultant first pool of cells is apportioned into a second set of portions each including multiple cells comprising first dual-indexed specific binding members. In other words, the first pool of cellular sources is divided or separated into multiple sub-portions, which collectively make up the second set of sub-portions, where the different sub-portions include a plurality of, or multiple, cellular sources, where the multiple cellular sources making up the different sub-portions include first identifier tagged nucleic acids, e.g., as described. above. While the number of portions making up the second plurality may vary, in some instances the number ranges from 2 to 25,000 portions, such as 10 to 10,000 portions, and including 20 to 1,000 portions, e.g., 50 to 500 portions, for example, 96 to 384 portions. In some instances, the number of portions in the second plurality is the same as the number of portions in the first plurality. As indicated above, each portion of the second plurality is made up of, i.e., includes, a plurality of cellular sources. While the number of cellular sources making up a given sub-portion of the second set may vary, in some instances the number ranges from 1 to 10,000, such as 100 to 1,000 and including from 100 to 500.

Within a given portion of the second plurality, multiple cellular sources making up that portion differ from each other with respect to the first dual-indexed specific binding member associated with cells of the portion. Because of the pooling/reapportioning step, cells from different portions of the first plurality are combined into the same portion of the second plurality. Within this same portion, the first dual-indexed specific binding members of the cells differ from each other with respect to at least the barcode domain of the dual-indexed specific binding members. As such, a given portion of the second plurality will have a plurality of different dual-indexed specific binding members associated therewith, with each different dual-indexed specific binding member associated with its own cellular source.

In embodiments, following production of the plurality of second portions, the methods include combining different portions of the second plurality with distinct second dual-indexed specific binding members having distinct fluorescence and oligonucleotide barcodes to stably associate cells of the portions with second dual-indexed specific binding members. The step may be performed as described above with respect to contact of the first plurality of portions with first dual-indexed specific binding members.

As indicated above, a given combinatorial, e.g., split/pool, indexing protocol may include two or more split/pool iterations. As such, in some instances the methods include splitting the second pool into a third plurality of portions; and combining different portions of the third plurality with distinct third dual-indexed specific binding members having distinct fluorescence and oligonucleotide barcodes to stably associate cells of the portions with third dual-indexed specific binding members.

The above methods result in the production of an indexed population of cells. The indexed population of cells is made up of a plurality of cells, where each uniquely indexed cell of the plurality has stably associated therewith its own unique combination of dual-indexed specific binding members. As such, different uniquely indexed cells have combinations of dual-indexed specific binding members associated therewith that different from any other combination of dual-indexed specific binding members associated with any other cell of the plurality.

### Phenotypic Labeling

In certain embodiments of the present invention, the methods may include detection of one or more phenotype characteristics of the cells. Detectable phenotypic characteristics include, but are not limited to, presence of an analyte, e.g., cell surface or internal marker, physical characteristic (e.g., size, shape, granularity, etc.), cell number (or frequency), etc. Virtually any detectable characteristic of interest can be assayed for as the detectable phenotypic characteristic of interest. In certain embodiments, the methods of the present invention are drawn to detecting the presence of an analyte, e.g., a marker, associated with (e.g., in, on, or attached to) the cells being assayed, either qualitatively or quantitatively. In some instances, a marker employed in phenotypic labeling is not a marker to which a cellular association member of a dual-indexed specific binding member binds, e.g., as described above.

In certain of these embodiments, the method includes contacting the indexed cell sample with a detectable analyte-specific binding agent. By “analyte-specific binding agent” and grammatical equivalents thereof, is meant any molecule, e.g., nucleic acids, small organic molecules, and proteins, nucleic acid binding dye (e.g., ethidium bromide) which are capable of associating with a specific analyte (or specific isoform of an analyte) in a cell over any others. Analytes of interest include any molecule associated with or present within the cells being analyzed in the subject methods. As such, analytes of interest include, but are not limited to, proteins, carbohydrates, organelles, nucleic acids, infectious particles (e.g., viruses, bacteria, parasites), metabolites, etc. In certain embodiments, the analyte-specific binding agent is a protein. In certain of these embodiments, the analyte-specific binding agent is an antibody or binding fragment thereof, e.g., as described above. Accordingly, the methods and compositions of the present invention may be used to detect any particular element isoform in a sample that is antigenically detectable and antigenically distinguishable from other isoforms of the activatable element that are present in the sample.

In certain embodiments, multiple detectable analyte-specific binding agents are employed in a method in accordance with the present invention. By “multiple analyte-specific binding agents” is meant that at least 2 or more analyte-specific binding agents are used, including 3 or more, 4 or more, 5 or more, etc. In certain embodiments, each of the different analyte-specific binding agents are labeled (again, either directly or indirectly) with a distinctly detectable label (e.g., fluorophores that have emission wavelengths that can be detected in distinct channels on a flow cytometer, with or without compensation). The multiple analyte-specific binding agents can bind to the same analyte in or on a cell (e.g., two antibodies that bind to different epitopes on the same protein), to different analytes in or on the cell, or in any combination (e.g., two agents that bind the same analyte and a third that binds to a distinct analyte). The upper limit for the number of analyte specific binding agents will depend largely on the parameters of the assay and the detection capacity of the detecting system employed. Where the analytes of interest are intracellular, indexed cells may be permeabilized, e.g., using protocols known in the art.

### Protein Expression Analysis

In some instances, a given workflow assesses protein expression, which may be assessed in combination with gene pression. In such embodiments, indexed cells may be contacted with a phenotypic biomarker label, e.g., a phenotypic biomarker specific binding member, e.g., antibody or binding fragment thereof, where the phenotypic biomarker label is conjugated to a phenotypic biomarker identifying oligonucleotide, e.g., an oligonucleotide that includes a barcode domain that identifies the specific binding member and phenotypic biomarker, e.g., antigen, to which it specifically binds. For example, embodiments of the methods may include contacting the indexed cells with one or more AbSeq antibody-oligonucleotide conjugates (Becton, Dickinson and Company), which are a commercially available example of phenotypic biomarker label-oligonucleotide conjugates. Further details regarding aspects of such phenotypic biomarker label-oligonucleotide conjugates as well as their use are provided in published PCT application publication nos. WO/2022/109343; WO/2021/163374; WO/2021/146207; WO/2020/159757; WO/2018/058073; as well as “Shahi et al., “Abseq: Ultrahigh-throughput single cell protein profiling with droplet microfluidic barcoding. Sci Rep 7, 44447 (2017),” the disclosures of which are herein incorporated by reference.

### Obtaining Flow Cytometric Data

Following production of a indexed composition, e.g., as described above, the methods may include flow cytometrically assaying the indexed composition. By “flow cytometrically assaying” is meant performing a flow cytometric assay on a composition, e.g., an assay composition, as described above. The flow cytometric assaying may include characterizing a sample, e.g., a sample including the assay composition, with a flow cytometer system. The flow cytometric assaying may include introducing the assay composition into a flow cytometer. A flow cytometer typically includes a sample reservoir for receiving a fluid sample, such as a sample including the assay composition, and a sheath reservoir containing a sheath fluid. The flow cytometer transports the particles (including cells, e.g., from the assay composition) in the fluid sample as a cell stream to a flow cell, while also directing the sheath fluid to the flow cell. To characterize the components of the flow stream, the flow stream is irradiated with light. Variations in the materials in the flow stream, such as morphologies or the presence of fluorescent labels, may cause variations in the observed light and these variations allow for characterization and separation. For example, particles, such as molecules, analyte-bound beads, or individual cells, in a fluid suspension are passed by a detection region in which the particles are exposed to an excitation light, typically from one or more lasers, and the light scattering and fluorescence properties of the particles are measured. Particles or components thereof typically are labeled with fluorescent dyes to facilitate detection. A multiplicity of different particles or components may be simultaneously detected by using spectrally distinct fluorescent dyes to label the different particles or components. In some implementations, a multiplicity of detectors, one for each of the scatter parameters to be measured, and one or more for each of the distinct dyes to be detected are included in the analyzer. For example, some embodiments include spectral configurations where more than one sensor or detector is used per dye. The data obtained include the signals measured for each of the light scatter detectors and the fluorescence emissions. In certain embodiments, the flow cytometric assay may detect a signal indicating the presence of the labeled secondary antibody in the sample. Where a signal is detected, the sample may include an antibody (antibodies) to the antigenic determinant of the coronaviral antigen.

As summarized above, a sample (e.g., in a flow stream of the flow cytometer) may be irradiated with light from a light source. In some embodiments, the light source is a broadband light source, emitting light having a broad range of wavelengths, such as for example, spanning 50 nm or more, such as 100 nm or more, such as 150 nm or more, such as 200 nm or more, such as 250 nm or more, such as 300 nm or more, such as 350 nm or more, such as 400 nm or more and including spanning 500 nm or more. For example, one suitable broadband light source emits light having wavelengths from 200 nm to 1500 nm. Another example of a suitable broadband light source includes a light source that emits light having wavelengths from 400 nm to 1000 nm. Where methods include irradiating with a broadband light source, broadband light source protocols of interest may include, but are not limited to, a halogen lamp, deuterium arc lamp, xenon arc lamp, stabilized fiber-coupled broadband light source, a broadband LED with continuous spectrum, super-luminescent emitting diode, semiconductor light emitting diode, wide spectrum LED white light source, an multi-LED integrated white light source, among other broadband light sources or any combination thereof.

In other embodiments, the methods include irradiating with a narrow band light source emitting a particular wavelength or a narrow range of wavelengths, such as for example with a light source which emits light in a narrow range of wavelengths like a range of 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light (i.e., monochromatic light). Where methods include irradiating with a narrow band light source, narrow band light source protocols of interest may include, but are not limited to, a narrow wavelength LED, laser diode or a broadband light source coupled to one or more optical bandpass filters, diffraction gratings, monochromators or any combination thereof.

In certain embodiments, methods include irradiating the sample with one or more lasers. As discussed above, the type and number of lasers will vary depending on the sample as well as desired light collected and may be a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCl) excimer laser or xenon-fluorine (XeF) excimer laser or a combination thereof. In other instances, the methods include irradiating the flow stream with a dye laser, such as a stilbene, coumarin or rhodamine laser. In yet other instances, methods include irradiating the flow stream with a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, methods include irradiating the flow stream with a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulim YAG laser, ytterbium YAG laser, ytterbium₂O₃ laser or cerium doped lasers and combinations thereof.

The sample may be irradiated with one or more of the above-mentioned light sources, such as 2 or more light sources, such as 3 or more light sources, such as 4 or more light sources, such as 5 or more light sources and including 10 or more light sources. The light source may include any combination of types of light sources. For example, in some embodiments, the methods include irradiating the sample in the flow stream with an array of lasers, such as an array having one or more gas lasers, one or more dye lasers and one or more solid-state lasers. Where desired, at least one laser will be used for excitation of the fluorescent barcodes, and other lasers for other fluorophores associated with the cells.

In certain instances, the flow stream is irradiated with a plurality of beams of frequency-shifted light and a cell in the flow stream is imaged by fluorescence imaging using radiofrequency tagged emission (FIRE) to generate a frequency-encoded image, such as those described in Diebold, et al. Nature Photonics Vol. 7(10); 806-810 (2013), as well as described in U.S. Pat. Nos. 9,423,353; 9,784,661; 9,983,132; 10,006,852; 10,078,045; 10,036,699; 10,222,316; 10,288,546; 10,324,019; 10,408,758; 10,451,538; 10,620,111; and U.S. Patent Publication Nos. 2017/0133857; 2017/0328826; 2017/0350803; 2018/0275042; 2019/0376895 and 2019/0376894 the disclosures of which are herein incorporated by reference. In such instances, flow cytometric data may include image data of the cells of the composition being assayed. (See e.g., Schraivogel et al., Science Vol. 375(6578); 315-320 (2022)).

Aspects of the present methods include collecting fluorescent light with a fluorescent light detector. A fluorescent light detector may, in some instances, be configured to detect fluorescence emissions from fluorescent molecules, e.g., labeled specific binding members (such as labeled antibodies that specifically bind to markers of interest) associated with the particle in the flow cell. In certain embodiments, methods include detecting fluorescence from the sample with one or more fluorescent light detectors, such as 2 or more, such as 3 or more, such as 4 or more, such as 5 or more, such as 6 or more, such as 7 or more, such as 8 or more, such as 9 or more, such as 10 or more, such as 15 or more and including 25 or more fluorescent light detectors. In embodiments, each of the fluorescent light detectors is configured to generate a fluorescence data signal. Fluorescence from the sample may be detected by each fluorescent light detector, independently, over one or more of the wavelength ranges of 200 nm-1200 nm. In some instances, methods include detecting fluorescence from the sample over a range of wavelengths, such as from 200 nm to 1200 nm, such as from 300 nm to 1100 nm, such as from 400 nm to 1000 nm, such as from 500 nm to 900 nm and including from 600 nm to 800 nm. In other instances, methods include detecting fluorescence with each fluorescence detector at one or more specific wavelengths. For example, the fluorescence may be detected at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof, depending on the number of different fluorescent light detectors in the subject light detection system. In certain embodiments, methods include detecting wavelengths of light which correspond to the fluorescence peak wavelength of certain fluorophores present in the sample. In embodiments, fluorescent flow cytometer data is received from one or more fluorescent light detectors (e.g., one or more detection channels), such as 2 or more, such as 3 or more, such as 4 or more, such as 5 or more, such as 6 or more and including 8 or more fluorescent light detectors (e.g., 8 or more detection channels).

Light from the sample may be measured at one or more wavelengths of, such as at 5 or more different wavelengths, such as at 10 or more different wavelengths, such as at 25 or more different wavelengths, such as at 50 or more different wavelengths, such as at 100 or more different wavelengths, such as at 200 or more different wavelengths, such as at 300 or more different wavelengths and including measuring the collected light at 400 or more different wavelengths.

In certain embodiments, the methods include spectrally resolving the light from each fluorophore of the fluorophore-biomolecule reagent pairs in the sample. In some embodiments, the overlap between each different fluorophore is determined and the contribution of each fluorophore to the overlapping fluorescence is calculated. In some embodiments, spectrally resolving light from each fluorophore includes calculating a spectral unmixing matrix for the fluorescence spectra for each of the plurality of fluorophores having overlapping fluorescence in the sample detected by the light detection system. In certain instances, spectrally resolving the light from each fluorophore and calculating a spectral unmixing matrix for each fluorophore may be used to estimate the abundance of each fluorophore, such as for example to resolve the abundance of target cells in the sample.

In certain embodiments, the methods include spectrally resolving light detected by a plurality of photodetectors such as described e.g., U.S. Pat. No. 11,009,400; U.S. Patent Application Publication Nos. 20210247293 and 20210325292; the disclosures of which are herein incorporated by reference in their entirety. For example, spectrally resolving light detected by the plurality of photodetectors of the second set of photodetectors may be include solving a spectral unmixing matrix using one or more of: 1) a weighted least square algorithm; 2) a Sherman-Morrison iterative inverse updater; 3) an LU matrix decomposition, such as where a matrix is decomposed into a product of a lower-triangular (L) matrix and an upper-triangular (U) matrix; 4) a modified Cholesky decomposition; 5) by QR factorization; and 6) calculating a weighted least squares algorithm by singular value decomposition. In certain embodiments, methods further include characterizing the spillover spreading of the light detected by a plurality of photodetectors such as described e.g., in U.S. Patent Application Publication No. 20210349004, the disclosure of which is herein incorporated by reference.

In certain instances, the abundance of fluorophores associated with (e.g., chemically associated (i.e., covalently, ionically) or physically associated) a target particle is calculated from the spectrally resolved light from each fluorophore associated with the particle. For instance, in one example the relative abundance of each fluorophore associated with a target particle is calculated from the spectrally resolved light from each fluorophore. In another example, the absolute abundance of each fluorophore associated with the target particle is calculated from the spectrally resolved light from each fluorophore. In certain embodiments, a particle may be identified or classified based on the relative abundance of each fluorophore determined to be associated with the particle. In these embodiments, the particle may be identified or classified by any convenient protocol such as by: comparing the relative or absolute abundance of each fluorophore associated with a particle with a control sample having particles of known identity; or by conducting spectroscopic or other assay analysis of a population of particles (e.g., cells) having the calculated relative or absolute abundance of associated fluorophores.

In certain embodiments, methods include sorting one or more of the particles (e.g., cells) of the sample that are identified based on the estimated abundance of the fluorophores associated with the particle. The term “sorting” is used herein in its conventional sense to refer to separating components (e.g., droplets containing cells, droplets containing non-cellular particles such as biological macromolecules) of a sample and in some instances, delivering the separated components to one or more sample collection containers. For example, methods may include sorting 2 or more components of the sample, such as 3 or more components, such as 4 or more components, such as 5 or more components, such as 10 or more components, such as 15 or more components and including sorting 25 or more components of the sample. In sorting particles identified based on the abundance of fluorophores associated with the particle, methods include data acquisition, analysis and recording, such as with a computer, where multiple data channels record data from each detector used in obtaining the overlapping spectra of the plurality of fluorophore-biomolecule reagent pairs associated with the particle. In these embodiments, analysis includes spectrally resolving light (e.g., by calculating the spectral unmixing matrix) from the plurality of fluorophores of the fluorophore-biomolecule reagent pairs having overlapping spectra that are associated with the particle and identifying the particle based on the estimated abundance of each fluorophore associated with the particle. This analysis may be conveyed to a sorting system which is configured to generate a set of digitized parameters based on the particle classification. In some embodiments, methods for sorting components of a sample include sorting particles (e.g., cells in a biological sample), such as described in U.S. Pat. Nos. 3,960,449; 4,347,935; 4,667,830; 5,245,318; 5,464,581; 5,483,469; 5,602,039; 5,643,796; 5,700,692; 6,372,506 and 6,809,804, the disclosures of which are herein incorporated by reference. In some embodiments, methods include sorting components of the sample with a particle sorting module, such as those described in U.S. U.S. Pat. Nos. 9,551,643 and 10,324,019, U.S. Patent Publication No. 2017/0299493 and International Patent Publication No. WO/2017/040151, the disclosure of which is incorporated herein by reference. In certain embodiments, cells of the sample are sorted using a sort decision module having a plurality of sort decision units, such as those described in U.S. Pat. No. 11,085,868, the disclosure of which is incorporated herein by reference.

Flow cytometric assay procedures are well known in the art. See, e.g., Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain aspects, flow cytometrically assaying the composition involves using a flow cytometer capable of simultaneous excitation and detection of multiple fluorophores, such as a BD Biosciences FACSCanto™ flow cytometer, used substantially according to the manufacturer's instructions. Methods of the present disclosure may involve image cytometry, such as is described in Holden et al. (2005) Nature Methods 2:773 and Valet, et al. 2004 Cytometry 59:167-171, the disclosures of which are incorporated herein by reference.

Suitable flow cytometry systems may include, but are not limited to, those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain instances, flow cytometry systems of interest include BD Biosciences FACSCanto™ flow cytometer, BD Biosciences FACSCanto™ II flow cytometer, BD Accuri™ flow cytometer, BD Accuri™ C6 Plus flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer, BD Biosciences LSRFortessa™ flow cytometer, BD Biosciences LSRFortessa™ X-20 flow cytometer, BD Biosciences FACSPresto™ flow cytometer, BD Biosciences FACSVia™ flow cytometer and BD Biosciences FACSCalibur™ cell sorter, a BD Biosciences FACSCount™ cell sorter, BD Biosciences FACSLyric™ cell sorter, BD Biosciences Via™ cell sorter, BD Biosciences Influx™ cell sorter, BD Biosciences Jazz™ cell sorter, BD Biosciences Aria™ cell sorter, BD Biosciences FACSAria™ II cell sorter, BD Biosciences FACSAria™ III cell sorter, BD Biosciences FACSAria™ Fusion cell sorter and BD Biosciences FACSMelody™ cell sorter, BD Biosciences FACSymphony™ S6 cell sorter or the like.

In some embodiments, the subject systems are flow cytometric systems, such those described in U.S. Pat. Nos. 10,663,476; 10,620,111; 10,613,017; 10,605,713; 10,585,031; 10,578,542; 10,578,469; 10,481,074; 10,302,545; 10,145,793; 10,113,967; 10,006,852; 9,952,076; 9,933,341; 9,726,527; 9,453,789; 9,200,334; 9,097,640; 9,095,494; 9,092,034; 8,975,595; 8,753,573; 8,233,146; 8,140,300; 7,544,326; 7,201,875; 7,129,505; 6,821,740; 6,813,017; 6,809,804; 6,372,506; 5,700,692; 5,643,796; 5,627,040; 5,620,842; 5,602,039; 4,987,086; 4,498,766; the disclosures of which are herein incorporated by reference in their entirety.

In some embodiments, the subject systems are particle sorting systems that are configured to sort particles with an enclosed particle sorting module, such as those described in U.S. Patent Publication No. 2017/0299493, the disclosure of which is incorporated herein by reference. In certain embodiments, particles (e.g., cells) of the sample are sorted using a sort decision module having a plurality of sort decision units, such as those described in U.S. Patent Publication No. 2020/0256781, the disclosure of which is incorporated herein by reference. In some embodiments, the subject systems include a particle sorting module having deflector plates, such as described in U.S. Patent Publication No. 2017/0299493, filed on Mar. 28, 2017, the disclosure of which is incorporated herein by reference.

In certain instances, flow cytometry systems of the invention are configured for imaging particles in a flow stream by fluorescence imaging using radiofrequency tagged emission (FIRE), such as those described in Diebold, et al. Nature Photonics Vol. 7(10); 806-810 (2013) as well as described in U.S. Pat. Nos. 9,423,353; 9,784,661; 9,983, 132; 10,006,852; 10,078,045; 10,036,699; 10,222,316; 10,288,546; 10,324,019; 10,408,758; 10,451,538; 10,620,111; and U.S. Patent Publication Nos. 2017/0133857; 2017/0328826; 2017/0350803; 2018/0275042; 2019/0376895 and 2019/0376894 the disclosures of which are herein incorporated by reference. FIG. 4 provides a schematic illustration of obtaining image comprising flow cytometric data via a FIRE protocol, e.g., with a FACSDiscover flow cytometer, such as described in Schraivogel et al., Science Vol. 375(6578); 315-320 (2022), of a labeled cell in accordance with embodiments of the invention. As illustrated, image data may be obtained from fluorescent barcodes provided by fluorophores that have little impact on other detector changes, such as barcodes provided by Horizon™ conjugated polymeric dyes BB515, BB550 and BB790 (BD Biosciences).

As discussed above, the method includes cytometric analysis which may include sorting. Cells of interest identified in the sample may be sorted and subsequently analyzed by any convenient analysis technique. Subsequent analysis techniques of interest include, but are not limited to, sequencing; assaying by CellSearch, as described in Food and Drug Administration (2004) Final rule. Fed Regist 69:26036-26038; assaying by CTC Chip, as described in Nagrath, et al. (2007) Nature 450:1235-1239; assaying by MagSweeper, as described in Talasaz, et al. (2009). Proc Natl Acad Sci USA 106:3970-3975; and assaying by nanostructured substrates, as described in Wang S, et al. (2011) Angew Chem Int Ed Engl 50: 3084-3088; the disclosures of which are incorporated herein by reference. Where desired, the sorting protocol may include distinguishing viable and dead cells, where any convenient staining protocol for identifying such cells may be incorporated into the methods. Of interest is certain embodiments is cytometry data obtained with the BD FACSDiscover™ S8 Cell Sorter with BD CellView™ Image Technology (BD Biosciences).

Analysis of the data acquired from a indexed sample of the invention involves analyzing the cells for the detectable characteristic(s) of interest (e.g., as described in greater detail above). Analysis of the detectable characteristic may be done at any convenient step in the data analysis phase, including before, during or after deconvolution. Indeed, because the acquired data can be analyzed and re-analyzed at will, no limitation with regard to the order of deconvolution and analysis of the detectable characteristic(s) is intended. For cells of interest, obtained data may include the fluorescent signature of the cells, e.g., as provided by the dual indexed specific binding members associated with the cells (as described above); as well as other characteristics of the cells, including markers associated with the cells, images of the cells, etc. The data may be provided in any convenient format, e.g., Flow Cytometry Standard (FCS) file format.

### Obtaining Sequence Data for Indexed Single Cells

Following obtaining of cytometric data for the indexed population of cells, e.g., as described above, methods of embodiments of invention may include obtaining sequence data for indexed cells of the sample (where sequence data may be obtained for all of the cells of a labeled sample or a portion of the cells of the labeled sample, e.g., cells of interest of a labeled sample, such as sorted cells obtained via the cytometric step. Sequence data may be obtained using any convenient protocol. In some instances, sequence data may be obtained using a protocol that includes partitioning the indexed cells, followed by generation of sequenceable libraries of nucleic acids obtained from the partitioned cells and then reading the sequenceable libraries.

### Partitioning Indexed Cells

Following production of indexed cells (i.e., cells stably associated with dual-indexed specific binding members, e.g., as described), embodiments of the methods include partitioning the indexed cells to produce partitioned indexed single cells each having dual-indexed specific binding members associated therewith. In some instances, the partitioning includes distributing the indexed cells into partitions or compartments so that compartments include single indexed cells. By “partitioning” is meant that the indexed cells are placed into small reaction chambers, which may be fluidically isolated structures defined by solid materials, such as microwells, configured to accommodate the indexed cells. In some embodiments of the disclosed methods, devices, and systems, a plurality of microwells that are randomly distributed across a substrate are used. In some embodiments, the plurality of microwells are distributed across a substrate in an ordered pattern, e.g. an ordered array. In some embodiments, a plurality of microwells are distributed across a substrate in a random pattern, e.g., a random array. The microwells can be fabricated in a variety of shapes and sizes. Appropriate well geometries include, but are not limited to, cylindrical, elliptical, cubic, conical, hemispherical, rectangular, or polyhedral, e.g., three dimensional geometries comprised of several planar faces, for example, rectangular cuboid, hexagonal columns, octagonal columns, inverted triangular pyramids, inverted square pyramids, inverted pentagonal pyramids, inverted hexagonal pyramids, or inverted truncated pyramids. In some embodiments, non-cylindrical microwells, e.g., wells having an elliptical or square footprint, may offer advantages in terms of being able to accommodate larger cells. In some embodiments, the upper and/or lower edges of the well walls may be rounded to avoid sharp corners and thereby decrease electrostatic forces that may arise at sharp edges or points due to concentration of electrostatic fields. Thus, use of rounded off corners may improve the ability to retrieve beads from the microwells. Microwell dimensions may be characterized in terms of absolute dimensions. In some instances, the average diameter of the microwells may range from about 5 μm to about 100 μm. In other embodiments, the average microwell diameter is at least 5 μm, at least 10 μm, at least 15 μm, at least 20 μm, at least 25 μm, at least 30 μm, at least 35 μm, at least 40 μm, at least 45 μm, at least 50 μm, at least 60 μm, at least 70 μm, at least 80 μm, at least 90 μm, or at least 100 μm. In yet other embodiments, the average microwell diameter is at most 100 μm, at most 90 μm, at most 80 μm, at most 70 μm, at most 60 μm, at most 50 μm, at most 45 μm, at most 40 μm, at most 35 μm, at most 30 μm, at most 25 μm, at most 20 μm, at most 15 μm, at most 10 μm, or at most 5 μm. The volumes of the microwells used in the methods of the invention may vary, ranging in some instances from about 200 μm³ to about 800,000 μm³. In some embodiments, the micro well volume is at least 200 μm³, at least 500 μm³, at least 1,000 μm³, at least 10,000 μm³, at least 25,000 μm³, at least 50,000 μm³, at least 100,000 μm³, at least 200,000 μm³, at least 300,000 μm³, at least 400,000 μm³, at least 500,000 μm³, at least 600,000 μm³, at least 700,000 μm³, or at least 800,000 μm³. In other embodiments, the microwell volume is at most 800,000 μm³, at most 700,000 μm³, at most 600,000 μm³, 500,000 μm³, at most 400,000 μm³, at most 300,000 μm³, at most 200,000 μm³, at most 100,000 μm³, at most 50,000 μm³, at most 25,000 μm³, at most 10,000 μm³, at most 1,000 μm³, at most 500 μm³, or at most 200 μm³. The number of microwells in a given device employed in embodiments of the invention may vary, where in some instances the number is 100 or more, such as 250 or more, e.g., 500 or more, including 1000 or more, such as 5,000 or more, e.g., 10,000 or more, wherein some instances the number is 15,000 or less, e.g., 12,500 or less. Microwells suitable for use in embodiments of the invention are further described in PCT application serial no. PCT/US2016/014612 published as WO/2016/118915, the disclosure of which is herein incorporated by reference. As used herein, a substrate can refer to a type of solid support. A substrate can, for example, comprise a plurality of microwells. For example, a substrate can be a well array comprising two or more microwells. In some embodiments, a microwell can comprise a small reaction chamber of defined volume. In some embodiments, a microwell can entrap one or more cells. In some embodiments, a microwell can entrap only one cell. In some embodiments, a microwell can entrap one or more solid supports. In some embodiments, a microwell can entrap only one solid support. In some embodiments, a microwell entraps a single cell and a single solid support (e.g., a bead). While the number of wells, e.g., microwells, in a well plate, e.g., microwell array, may vary in a given apportioning step, in some instances the number ranged from 5 to 500, such as 5 to 100.

In partitioning indexed cells, the indexed cells may be positioned in compartments, e.g., microwells of a microwell array, using any convenient protocol. The disclosure provides for methods for compartmenting the indexed cells into partitions in order to partition the indexed cells. A collection of indexed cells, for example, can be introduced into structures, e.g., microwells, to partition the indexed cells. The indexed cells can be contacted, for example, by gravity flow wherein indexed cells can settle into the partitioning structures. In some instances, an aqueous composition of the indexed cells is contacted with, e.g., by flowing it across, an array of microwells such that indexed cells are deposited into the microwells. The aqueous composition that includes the indexed cells may be flowed through a flow cell in fluidic communication with the microwells. Suitable protocols and systems for partitioning the capture particles into microwells are described in Microwells suitable for use in embodiments of the invention are further described in PCT application serial no. PCT/US2016/014612 published as WO/2016/118915, the disclosure of which is herein incorporated by reference. To partition the cells of the cell sample, any convenient protocol may be employed, e.g., dispensing, such as pipetting, aliquots of the cellular sample into the compartments, flowing sample over the surface of the well plate, etc.

In some embodiments, partitioning a plurality of indexed cells further includes providing a particle, e.g., bead, that includes a particle, e.g., bead, bound nucleic acid into partitions that include the single cells, where the bound nucleic acid is employed in preparing nucleic acid sequence ready compositions, e.g., sequence ready libraries, from the labeled cells. In some instances, the particle, e.g., bead, bound nucleic acid includes a target binding region, e.g., that binds to complementary sequences in nucleic acid species of interest in the cell as well as to capture sequences of the dual indexed beads. For example, where target nucleic acids species are cellular mRNA and the dual-indexed specific binding member oligonucleotide barcodes include a poly(A) capture sequence, a bead bound nucleic acid may include a poly (T) domain as a target binding region. In addition to the target binding region, the bound nucleic acid may further include one or more additional domains, such as but not limited to: cell label domains, barcode domains, molecular index domains (e.g., unique molecular identifier (UMI) domain), universal primer binding domains, etc. Further details regarding particles having bound nucleic acids that may be provided in compartments may be found in in U.S. Patent Application Publication No. US2018/0088112; US Patent Application Publication No. 2018/0200710; U.S. Patent Application Publication No. US2018/0346970; U.S. Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference. Beads having bound nucleic acids may be provided in the compartments using any convenient protocol, including but not limited to, those described above for partitioning of cells, and further described in further described in PCT application serial no. PCT/US2016/014612 published as WO/2016/118915, the disclosure of which is herein incorporated by reference. The particles, e.g., beads, may be partitioned into the cells before or after, or in some instances in combination with, the indexed cells, as desired.

### Sequenceable Library Generation

Partitioning of the indexed cells, e.g., as described above, results in partitioned labeled cells that are in spatial proximity to a particle, e.g., bead, having bound cell label domain nucleic acids that include a target binding region, e.g., as described above. When cell label domain nucleic acids are in close proximity to targets and/or dual-indexed specific binding member oligonucleotide barcodes of the indexed single cells, the targets/oligonucleotide barcodes can hybridize to the cell label domain nucleic acid. The cell label domain comprising nucleic acid can be contacted at a non-depletable ratio such that each distinct target can associate with a distinct cell label domain comprising nucleic acid having its own unique UMI, if so desired.

Following the partitioning of the indexed cells, as described above, the indexed cells can be lysed to liberate the target molecules so that the released target molecules, e.g., nucleic acids, can bind to the target binding regions of the cell label domain nucleic acids to produce captured nucleic acids. Cell lysis can be accomplished by any of a variety of means, for example, by chemical or biochemical means, by osmotic shock, or by means of thermal lysis, mechanical lysis, or optical lysis. Particles can be lysed by addition of a cell lysis buffer comprising a detergent (e.g., SDS, Li dodecyl sulfate, Triton X-100, Tween-20, or NP-40), an organic solvent (e.g., methanol or acetone), or digestive enzymes (e.g., proteinase K, pepsin, or trypsin), or any combination thereof. To increase the association of a target and a barcode, the rate of the diffusion of the target molecules can be altered by for example, reducing the temperature and/or increasing the viscosity of the lysate. In some embodiments, the sample can be lysed using a filter paper. The filter paper can be soaked with a lysis buffer on top of the filter paper. The filter paper can be applied to the sample with pressure which can facilitate lysis of the sample and hybridization of the targets of the sample to the substrate. In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about 0.01, 0.05, 0.1, 0.5, or 1 M or more Tris HCl. A lysis buffer can comprise at most about 0.01, 0.05, 0.1, 0.5, or 1 M or more Tris HCL. A lysis buffer can comprise about 0.1 M Tris HCl. The pH of the lysis buffer can be at least about 1, 2, 3, 4, 5, 6, 7, 8, 9, 10, or more. The pH of the lysis buffer can be at most about 1, 2, 3, 4, 5, 6, 7, 8, 9, 10, or more. In some embodiments, the pH of the lysis buffer is about 7.5. The lysis buffer can comprise a salt (e.g., LiCl). The concentration of salt in the lysis buffer can be at least about 0.1, 0.5, or 1 M or more. The concentration of salt in the lysis buffer can be at most about 0.1, 0.5, or 1 M or more. In some embodiments, the concentration of salt in the lysis buffer is about 0.5M. The lysis buffer can comprise a detergent (e.g., SDS, Li dodecyl sulfate, triton X, tween, NP-40). The concentration of the detergent in the lysis buffer can be at least about 0.0001%, 0.0005%, 0.001%, 0.005%, 0.01%, 0.05%, 0.1%, 0.5%, 1%, 2%, 3%, 4%, 5%, 6%, or 7%, or more. The concentration of the detergent in the lysis buffer can be at most about 0.0001%, 0.0005%, 0.001%, 0.005%, 0.01%, 0.05%, 0.1%, 0.5%, 1%, 2%, 3%, 4%, 5%, 6%, or 7%, or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about 1, 5, 10, 15, or 20 mM or more. The concentration of the reducing reagent in the lysis buffer can be at most about 1, 5, 10, 15, or 20 mM or more. In some embodiments, the concentration of reducing reagent in the lysis buffer is about 5 mM. In some embodiments, a lysis buffer can comprise about 0.1M TrisHCl, about pH 7.5, about 0.5M LiCl, about 1% lithium dodecyl sulfate, about 10 mM EDTA, and about 5 mM DTT. Lysis can be performed at a temperature of about 4, 10, 15, 20, 25, or 30 °C. Lysis can be performed for about 1, 5, 10, 15, or 20 or more minutes. A lysed cell can comprise at least about 100000, 200000, 300000, 400000, 500000, 600000, or 700000 or more target nucleic acid molecules. A lysed cell can comprise at most about 100000, 200000, 300000, 400000, 500000, 600000, or 700000 or more target nucleic acid molecules.

Following lysis of the indexed cells and release of nucleic acid molecules therefrom, the nucleic acid molecules can randomly associate with the cell label domain nucleic acids of the co-localized solid support, e.g., bead. Association can comprise hybridization of a cell label domain nucleic acid's target recognition region to a complementary portion of the target nucleic acid molecule (e.g., oligo(dT) of the barcode can interact with a poly(A) tail of a target). The assay conditions used for hybridization (e.g., buffer pH, ionic strength, temperature, etc.) can be chosen to promote formation of specific, stable hybrids. In some embodiments, the nucleic acid molecules released from the lysed cells can associate with the plurality of probes on the substrate (e.g., hybridize with the probes on the substrate). When the probes comprise oligo(dT), mRNA molecules can hybridize to the probes and be reverse transcribed. The oligo(dT) portion of the oligonucleotide can act as a primer for first strand synthesis of the cDNA molecule, e.g., when subject to DNA synthesis reaction conditions to produce first strand cDNA domain comprising capture nucleic acids. Cell label domain nucleic acid can also hybridize to complementary capture sequences of dual index oligonucleotide barcodes, e.g., poly(A) sequences, of the dual-indexed specific binding member oligonucleotide barcodes associated with the labeled cells. In this way, the cell label domain nucleic acids can act as primers for reverse transcription using the dual-indexed specific binding member oligonucleotide barcode as a template, e.g., as described in greater detail below.

Where desired, a given workflow may include a pooling step where a product composition, e.g., made up of captured nucleic acids, synthesized first strand cDNAs or synthesized double stranded cDNAs, is combined or pooled with product compositions obtained from one or more additional samples, e.g., labeled cells. In some instances, the pooling step is performed just after hybridization step between cell label domain nucleic acids and target nucleic acids, e.g., as reviewed above. The number of different product compositions produced from different samples, e.g., cells, that are combined or pooled in such embodiments may vary, where the number ranges in some instances from 2 to 1,000,000, such as 3 to 200,000, including 4 to 100,000 such as 5 to 50,000, where in some instances the number ranges from 100 to 10,000, such as 1,000 to 5,000. Prior to or after pooling, the product composition(s) can be amplified, e.g., by polymerase chain reaction (PCR), such as described in greater detail below. Once the target-cell domain label molecules have been pooled, all further processing can proceed in a single reaction vessel. Further processing can include, for example, reverse transcription reactions, amplification reactions, cleavage reactions, dissociation reactions, and/or nucleic acid extension reactions. Further processing reactions can be performed within the microwells, that is, without first pooling the labeled target nucleic acid molecules from a plurality of cells.

The disclosure provides for a method to create a target-cell label domain conjugate using any convenient protocol, such as reverse transcription or nucleotide extension. The target-cell label domain conjugate can comprise the cell label domain and a complementary sequence of all or a portion of the target nucleic acid. Reverse transcription of the associated RNA molecule can occur by the addition of a reverse transcription primer along with the reverse transcriptase. The reverse transcription primer can be an oligo(dT) primer, a random hexanucleotide primer, or a target-specific oligonucleotide primer. Oligo(dT) primers can be, or can be about, 12-18 nucleotides in length and bind to the endogenous poly(A) tail at the 3′ end of mammalian mRNA. Random hexanucleotide primers can bind to mRNA at a variety of complementary sites. Target-specific oligonucleotide primers typically selectively prime the mRNA of interest. Reverse transcription can occur repeatedly to produce multiple cDNA molecules. The methods disclosed herein can comprise conducting at least about 1, 2, 3, 4, 5, 6, 7, 8, 9, 10, 11, 12, 13, 14, 15, 16, 17, 18, 19, or 20 reverse transcription reactions. The method can comprise conducting at least about 25, 30, 35, 40, 45, 50, 55, 60, 65, 70, 75, 80, 85, 90, 95, or 100 reverse transcription reactions.

One or more nucleic acid amplification reactions can be performed to create multiple copies of the target nucleic acid molecules. Amplification can be performed in a multiplexed manner, wherein multiple target nucleic acid sequences are amplified simultaneously. The amplification reaction can be used to add sequencing adapters to the nucleic acid molecules. The amplification reactions can comprise amplifying at least a portion of a sample label, if present. The amplification reactions can comprise amplifying at least a portion of the cellular label and/or barcode sequence (e.g., a molecular label). The amplification reactions can comprise amplifying at least a portion of a sample tag, a cell label, a spatial label, a barcode sequence (e.g., a molecular label), a target nucleic acid, or a combination thereof. The amplification reactions can comprise amplifying 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 100%, or a range or a number between any two of these values, of the plurality of nucleic acids. The method can further comprise conducting one or more cDNA synthesis reactions to produce one or more cDNA copies of target-barcode molecules comprising a sample label, a cell label, a spatial label, and/or a barcode sequence (e.g., a molecular label).

In some embodiments, amplification can be performed using a polymerase chain reaction (PCR). As used herein, PCR can refer to a reaction for the in vitro amplification of specific DNA sequences by the simultaneous primer extension of complementary strands of DNA. As used herein, PCR can encompass derivative forms of the reaction, including but not limited to, RT-PCR, real-time PCR, nested PCR, quantitative PCR, multiplexed PCR, digital PCR, and assembly PCR.

Amplification of the nucleic acids can comprise non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), and a Qβ replicase (Qβ) method, use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and ramification extension amplification (RAM). In some embodiments, the amplification does not produce circularized transcripts.

In some embodiments, the methods disclosed herein further comprise conducting a polymerase chain reaction on the nucleic acid (e.g., RNA, DNA, cDNA) to produce a labeled amplicon (e.g., a stochastically labeled amplicon). The labeled amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample label, a spatial label, a cell label, and/or a barcode sequence (e.g., a molecular label). The labeled amplicon can be a single-stranded molecule. The single-stranded molecule can comprise DNA, RNA, or a combination thereof. The nucleic acids of the disclosure can comprise synthetic or altered nucleic acids. As such, methods may include producing an amplicon composition from the first strand cDNA domain comprising capture nucleic acids.

Amplification can comprise use of one or more non-natural nucleotides. Non-natural nucleotides can comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise, for example, 1, 2, 3, 4, 5, 6, 7, 8, 9, 10, 11, 12, 13, 14, or 15 or more nucleotides. The one or more primers can comprise at least 1, 2, 3, 4, 5, 6, 7, 8, 9, 10, 11, 12, 13, 14, or 15 or more nucleotides. The one or more primers can comprise less than 12-15 nucleotides. The one or more primers can anneal to at least a portion of the plurality of labeled targets (e.g., stochastically labeled targets). The one or more primers can anneal to the 3′ end or 5′ end of the plurality of labeled targets. The one or more primers can anneal to an internal region of the plurality of labeled targets. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled targets. The one or more primers can comprise a fixed panel of primers. The one or more primers can comprise at least one or more custom primers. The one or more primers can comprise at least one or more control primers. The one or more primers can comprise at least one or more gene-specific primers.

The one or more primers can comprise a universal primer. The universal primer can anneal to a universal primer binding site. The one or more custom primers can anneal to a first sample label, a second sample label, a spatial label, a cell label, a barcode sequence (e.g., a molecular label), a target, or any combination thereof. The one or more primers can comprise a universal primer and a custom primer. The custom primer can be designed to amplify one or more targets. The targets can comprise a subset of the total nucleic acids in one or more samples. The targets can comprise a subset of the total labeled targets in one or more samples. The one or more primers can comprise at least 96 or more custom primers. The one or more primers can comprise at least 960 or more custom primers. The one or more primers can comprise at least 9600 or more custom primers. The one or more custom primers can anneal to two or more different labeled nucleic acids. The two or more different labeled nucleic acids can correspond to one or more genes.

Any amplification scheme can be used in the methods of the present disclosure. For example, in one scheme, the first round PCR can amplify molecules attached to the bead using a gene specific primer and a primer against the universal Illumina sequencing primer 1 sequence. The second round of PCR can amplify the first PCR products using a nested gene specific primer flanked by Illumina sequencing primer 2 sequence, and a primer against the universal Illumina sequencing primer 1 sequence. The third round of PCR adds P5 and P7 and sample index to turn PCR products into an Illumina sequencing library. Sequencing using 150 bp×2 sequencing can reveal the cell label and barcode sequence (e.g., molecular label) on read 1, the gene on read 2, and the sample index on index 1 read.

In some embodiments, nucleic acids can be removed from the substrate using chemical cleavage. For example, a chemical group or a modified base present in a nucleic acid can be used to facilitate its removal from a solid support. For example, an enzyme can be used to remove a nucleic acid from a substrate. For example, a nucleic acid can be removed from a substrate through a restriction endonuclease digestion. For example, treatment of a nucleic acid containing a dUTP or ddUTP with uracil-d-glycosylase (UDG) can be used to remove a nucleic acid from a substrate. For example, a nucleic acid can be removed from a substrate using an enzyme that performs nucleotide excision, such as a base excision repair enzyme, such as an apurinic/apyrimidinic (AP) endonuclease. In some embodiments, a nucleic acid can be removed from a substrate using a photocleavable group and light. In some embodiments, a cleavable linker can be used to remove a nucleic acid from the substrate. For example, the cleavable linker can comprise at least one of biotin/avidin, biotin/streptavidin, biotin/neutravidin, Ig-protein A, a photo-labile linker, acid or base labile linker group, or an aptamer.

In some embodiments, amplification can be performed on the substrate, for example, with bridge amplification. cDNAs can be homopolymer tailed in order to generate a compatible end for bridge amplification using oligo(dT) probes on the substrate. In bridge amplification, the primer that is complementary to the 3′ end of the template nucleic acid can be the first primer of each pair that is covalently attached to the solid particle. When a sample containing the template nucleic acid is contacted with the particle and a single thermal cycle is performed, the template molecule can be annealed to the first primer and the first primer is elongated in the forward direction by addition of nucleotides to form a duplex molecule consisting of the template molecule and a newly formed DNA strand that is complementary to the template. In the heating step of the next cycle, the duplex molecule can be denatured, releasing the template molecule from the particle and leaving the complementary DNA strand attached to the particle through the first primer. In the annealing stage of the annealing and elongation step that follows, the complementary strand can hybridize to the second primer, which is complementary to a segment of the complementary strand at a location removed from the first primer. This hybridization can cause the complementary strand to form a bridge between the first and second primers secured to the first primer by a covalent bond and to the second primer by hybridization. In the elongation stage, the second primer can be elongated in the reverse direction by the addition of nucleotides in the same reaction mixture, thereby converting the bridge to a double-stranded bridge. The next cycle then begins, and the double-stranded bridge can be denatured to yield two single-stranded nucleic acid molecules, each having one end attached to the particle surface via the first and second primers, respectively, with the other end of each unattached. In the annealing and elongation step of this second cycle, each strand can hybridize to a further complementary primer, previously unused, on the same particle, to form new single-strand bridges. The two previously unused primers that are now hybridized elongate to convert the two new bridges to double-strand bridges. The amplification reactions can comprise amplifying at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the plurality of nucleic acids.

Amplification of the labeled nucleic acids can comprise PCR-based methods or non-PCR based methods. Amplification of the labeled nucleic acids can comprise exponential amplification of the labeled nucleic acids. Amplification of the labeled nucleic acids can comprise linear amplification of the labeled nucleic acids. Amplification can be performed by polymerase chain reaction (PCR). PCR can refer to a reaction for the in vitro amplification of specific DNA sequences by the simultaneous primer extension of complementary strands of DNA. PCR can encompass derivative forms of the reaction, including but not limited to, RT-PCR, real-time PCR, nested PCR, quantitative PCR, multiplexed PCR, digital PCR, suppression PCR, semi-suppressive PCR and assembly PCR.

In some embodiments, amplification of the labeled nucleic acids comprises non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), a Qβ replicase (Qβ), use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and/or ramification extension amplification (RAM).

In some embodiments, the methods disclosed herein further comprise conducting a nested polymerase chain reaction on the amplified amplicon (e.g., target). The amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample tag or molecular identifier label. Alternatively, the amplicon can be a single-stranded molecule. The single-stranded molecule can comprise DNA, RNA, or a combination thereof. The nucleic acids of the present invention can comprise synthetic or altered nucleic acids.

In some embodiments, the method comprises repeatedly amplifying the labeled nucleic acid to produce multiple amplicons. The methods disclosed herein can comprise conducting at least about 1, 2, 3, 4, 5, 6, 7, 8, 9, 10, 11, 12, 13, 14, 15, 16, 17, 18, 19, or 20 amplification reactions. Alternatively, the method comprises conducting at least about 25, 30, 35, 40, 45, 50, 55, 60, 65, 70, 75, 80, 85, 90, 95, or 100 amplification reactions.

Amplification can further comprise adding one or more control nucleic acids to one or more samples comprising a plurality of nucleic acids. Amplification can further comprise adding one or more control nucleic acids to a plurality of nucleic acids. The control nucleic acids can comprise a control label.

Amplification can comprise use of one or more non-natural nucleotides. Non-natural nucleotides can comprise photolabile and/or triggerable nucleotides. Examples of non-natural nucleotides include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise one or more oligonucleotides. The one or more oligonucleotides can comprise at least about 7-9 nucleotides. The one or more oligonucleotides can comprise less than 12-15 nucleotides. The one or more primers can anneal to at least a portion of the plurality of labeled nucleic acids. The one or more primers can anneal to the 3′ end and/or 5′ end of the plurality of labeled nucleic acids. The one or more primers can anneal to an internal region of the plurality of labeled nucleic acids. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled nucleic acids. The one or more primers can comprise a fixed panel of primers. The one or more primers can comprise at least one or more custom primers. The one or more primers can comprise at least one or more control primers. The one or more primers can comprise at least one or more housekeeping gene primers. The one or more primers can comprise a universal primer. The universal primer can anneal to a universal primer binding site. The one or more custom primers can anneal to the first sample tag, the second sample tag, the molecular identifier label, the nucleic acid or a product thereof. The one or more primers can comprise a universal primer and a custom primer. The custom primer can be designed to amplify one or more target nucleic acids. The target nucleic acids can comprise a subset of the total nucleic acids in one or more samples. In some embodiments, the primers are the probes attached to the array of the disclosure.

In some embodiments, barcoding (e.g., stochastically barcoding) the plurality of targets in the sample further comprises generating an indexed library of the barcoded targets (e.g., stochastically barcoded targets) or barcoded fragments of the targets. The barcode sequences of different barcodes (e.g., the molecular labels of different stochastic barcodes) can be different from one another. Generating an indexed library of the barcoded targets includes generating a plurality of indexed polynucleotides from the plurality of targets in the sample. For example, for an indexed library of the barcoded targets comprising a first indexed target and a second indexed target, the label region of the first indexed polynucleotide can differ from the label region of the second indexed polynucleotide by, by about, by at least, or by at most, 1, 2, 3, 4, 5, 6, 7, 8, 9, 10, 20, 30, 40, 50, or a number or a range between any two of these values, nucleotides. In some embodiments, generating an indexed library of the barcoded targets includes contacting a plurality of targets, for example mRNA molecules, with a plurality of oligonucleotides including a poly(T) region and a label region; and conducting a first strand synthesis using a reverse transcriptase to produce single-strand labeled cDNA molecules each comprising a cDNA region and a label region, wherein the plurality of targets includes at least two mRNA molecules of different sequences and the plurality of oligonucleotides includes at least two oligonucleotides of different sequences. Generating an indexed library of the barcoded targets can further comprise amplifying the single-strand labeled cDNA molecules to produce double-strand labeled cDNA molecules; and conducting nested PCR on the double-strand labeled cDNA molecules to produce labeled amplicons. In some embodiments, the method can include generating an adaptor-labeled amplicon.

Barcoding (e.g., stochastic barcoding) can include using nucleic acid barcodes or tags to label individual nucleic acid (e.g., DNA or RNA) molecules. In some embodiments, it involves adding DNA barcodes or tags to cDNA molecules as they are generated from mRNA. Nested PCR can be performed to minimize PCR amplification bias. Adapters can be added for sequencing using, for example, next generation sequencing (NGS). The sequencing results can be used to determine cell labels, molecular labels, and sequences of nucleotide fragments of the one or more copies of the targets.

### Sample Indexing

In some instances, embodiments of the methods may include sample indexing, e.g., where a given sample is to be combined with other samples for downstream processing, e.g., flow cytometric analysis, sequencing, etc. In such instances, a given indexed population of cells prepared from a first cellular sample may be contacted with a sample indexing reagent such that the sample indexing reagent becomes stably associated with indexed cells of the indexed population. While sample indexing reagents may vary, in some instances the sample indexing reagent may be a specific binding member sample identifying oligonucleotide conjugate, where these components may be as described above. In embodiments where protein expression is assayed, e.g., where phenotypic biomarker labels comprising a conjugated phenotypic biomarker identifying oligonucleotide are employed (such as described above), the phenotypic biomarker labels comprising a conjugated phenotypic biomarker identifying oligonucleotide may also be conjugated to a sample identifying oligonucleotide. For example, an AbSeq antibody including both an antibody identifying barcode and a sample indexing barcode may be employed. One or more additional indexed population of cells from one or more additional samples may be similarly contact with additional sample indexing reagents. Following preparation of the disparate indexed samples, the indexed samples may be combined or pooled for further processing, e.g., flow cytometric analysis, sequencing, etc., such as described in greater detail elsewhere in this application. Sample indexing protocols are further described in published PCT application publication nos. WO/2020/046833; WO/2020/037065; and WO/2018/226293; the disclosures of which are herein incorporated by reference. In some instances, a commercial sample multiplexing system may be employed, such as the BD™ Single-Cell Multiplexing Kit (Becton, Dickinson and Company).

### Sequencing

In certain embodiments, the methods provided further include subjecting a prepared expression library, e.g., an amplicon composition produced as described above, to a sequencing protocol, such as a Next Generation Sequence (NGS) protocol. The protocol may be carried out on any suitable NGS sequencing platform. NGS sequencing platforms of interest include, but are not limited to, a sequencing platform provided by Illumina® (e.g., the HiSeq™, MiSeq™ and/or NextSeq™ sequencing systems); Ion Torrent™ (e.g., the Ion PGM™ and/or Ion Proton™ sequencing systems); Pacific Biosciences (e.g., the PACBIO RS II Sequel sequencing system); Life Technologies™ (e.g., a SOLiD sequencing system); Oxford Nanopore (e.g., Minion), Roche (e.g., the 454 GS FLX₊ and/or GS Junior sequencing systems); or any other sequencing platform of interest. The NGS protocol will vary depending on the particular NGS sequencing system employed. Detailed protocols for sequencing, e.g., which may include further amplification (e.g., solid-phase amplification), sequencing the amplicons, and analyzing the sequencing data are available from the manufacturer of the NGS sequencing system employed.

In some instances, the methods further include employing oligonucleotide labeled cellular component binding reagents, e.g., in applications where detection, e.g., quantitation, of one of or more cellular components, e.g., surface proteins, is desired (e.g., via a BD AbSeq protocol). Oligonucleotide labeled cellular component-binding reagents employed in such embodiments include a cellular component-binding reagent, e.g., antibody or binding fragment thereof, coupled to a cellular component-binding reagent specific oligonucleotide comprising an identifier sequence for the cellular component-binding reagent that the cellular component-binding reagent specific oligonucleotide is associated therewith. In such instances, the magnetic capture bead may include a nucleic acid configured to capture, e.g., specifically bind to, a domain of the cellular component-binding reagent specific oligonucleotide. In this way, protein expression may be assayed in conjunction with gene expression, e.g., where multi-ohmic analysis is desired, e.g., combined analysis of transcriptome and proteome. In such instances, the methods may include preparing the captured sample with oligonucleotide labeled cellular component binding reagents, and then provide for capture of cellular component-binding reagent specific oligonucleotides released from the capture, partitioned cells. Further details regarding use of oligonucleotide labeled cellular component-binding reagents are found in United States Published Patent Application Nos. US20180267036 and US20200248263; the disclosures of which are herein incorporated by reference.

Further details regarding methods for obtaining sequence data from single cells, e.g., as described above, are provided in U.S. Patent Application Publication No. US2018/0088112; US Patent Application Publication No. 2018/0200710; U.S. Patent Application Publication No. US2018/0346970; U.S. Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference.

### Linking Cytometric Data and Sequence Data

The sequencing protocol generates sequence data for the labeled cells. This sequence data can then be readily linked to cytometric data for the labeled cells, such that cytometric data and sequence data obtained from the same cells may be paired. In other words, a given set of cytometric, e.g., image, data and a given set of sequence data may be linked as being obtained from the same cell, e.g., as described in greater detail below.

Following obtainment of cytometric data and sequence data, e.g., as described above, the obtained cytometric and sequence data obtained from a given cell, is linked. By linked is meant that cytometric and sequence data are paired as originating from the same cell. As such, cytometric data and sequence data obtained from the same labeled cells may be paired. In other words, a given set of cytometric data and a given set of sequence data may be identified as being obtained from the same cell and then paired or otherwise associated with each other. In this manner, linked cytometric and sequence data may be obtained for single cells of a cellular sample.

The cytometric data and sequence data is linked by using the fluorescent signature and oligonucleotide barcodes provided by the dual indexed specific binding members associated with the indexed cells from which the cytometric and sequence data is obtained. In the obtained sequence data, e.g., as described above, sequence reads for both cellular targets and dual-indexed specific binding member oligonucleotide barcodes of indexed cells are obtained. In other words, for each indexed cell assayed in a given work flow, the sequence of the dual indexed specific binding member oligonucleotide barcodes associated with that cell and the sequence of target nucleic acids from that cell, e.g., mRNAs from the cell, are obtained. For each indexed cell, these obtained sequences are obtained using a protocol (which may be a next generation sequencing protocol), such as described above, where a library is generated from the original sequences, where each member of a given library generated from the same partition shares a common cell label. As such, sequence reads from the cellular target nucleic acids and the dual-indexed specific binding member oligonucleotide barcodes that are obtained from the cell all share the same cell label, i.e., they all have a common cell label. In linking the cell and image data, all reads that have the same cell label domain, i.e., that share a common cell label, from both reads of target nucleic acids and reads of dual-indexed specific binding member oligonucleotide barcodes, may be paired or linked. This pairing or linkage results in a set of reads that includes reads of both target nucleic acids and dual-indexed specific binding member oligonucleotide barcode nucleic acids, and these reads can be identified as originating from the same cell.

Next, the resultant sequence data that includes reads of both target nucleic acids and dual-indexed specific binding member oligonucleotide barcode nucleic acids may be matched, i.e., paired or linked, with cytometric data. As reviewed above, cytometric data for indexed cells includes fluorescent signatures for those cells, where the fluorescent signatures are provided by the one or more dual-indexed specific binding members associated with those cells. When cells are cytometrically analyzed to obtain cytometric data therefor, a series or collection of fluorescent signals that is obtained from the dual-indexed specific binding members associated with those cells is obtained, where this series may be referred to as a cell specific fluorescent signature. Different cells of a given workflow will have their own unique cell specific fluorescent signature. A given fluorescent signal provided by a dual-indexed specific binding members making up such a cell specific fluorescent signature can be assigned to a given portion of a sequence read because the sequence of a dual-indexed specific binding member oligonucleotide barcodes from which that fluorescent signal is obtained is known. As such, each cell specific fluorescent signature obtained for a given labeled cell can be used to determine the sequences of the different dual-indexed specific binding member oligonucleotide barcodes associated with that indexed cell. As the sequences of the dual-indexed specific binding member oligonucleotide barcodes are present in the reads of the oligonucleotide barcodes, a given cell specific fluorescent signature may be determined as being associated with a given set of sequence data. Once a cell specific fluorescent signature is associated with the given set of sequence data, the sequence data can be determined as being obtained from the same indexed cell that was in that partition from which the cell specific fluorescent signature was obtained. In other words, from a series of fluorescent signals obtained from a given cell during cytometric analysis, a fluorescent signature may be obtained for that cell. Because a given fluorescent signature can be matched to reads from oligonucleotide barcodes from dual-indexed specific binding members, the fluorescent signature can be matched with sequence reads from the dual-indexed specific binding members that produced the fluorescent signature, where the matched sequence reads from the dual-indexed specific binding members may then be used to identify all sequence data obtained from a given partition and cell in that partition. Once the sequence data is assigned to a given partition, the sequence data may then be readily linked with cytometric data obtained for the cell in that partition. In this manner, linked cytometric and sequence data may be obtained for single cells of a cellular sample.

### Kits

Aspects of the invention further include kits and compositions that find use in practicing various embodiments of methods of the invention. Kits of the invention may include: a population of dual-indexed specific binding members; beads comprising a bead bound nucleic acid comprising a cell label domain and target binding region, e.g., as described above, and/or other reagents, as desired. The population of dual-indexed specific binding members may include a varying number of distinct fluorescent and oligonucleotide barcodes that differ from each other. While the number of distinct dual-indexed specific binding members of a given population may vary, in some instances the number ranges from 5 to 1,000, such as 10 to 500.

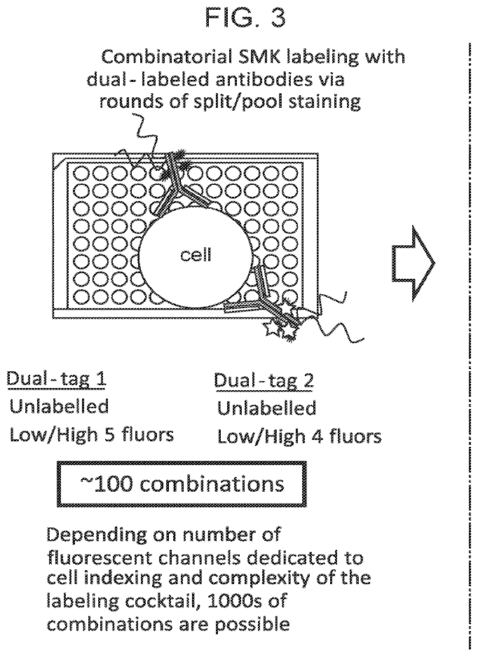

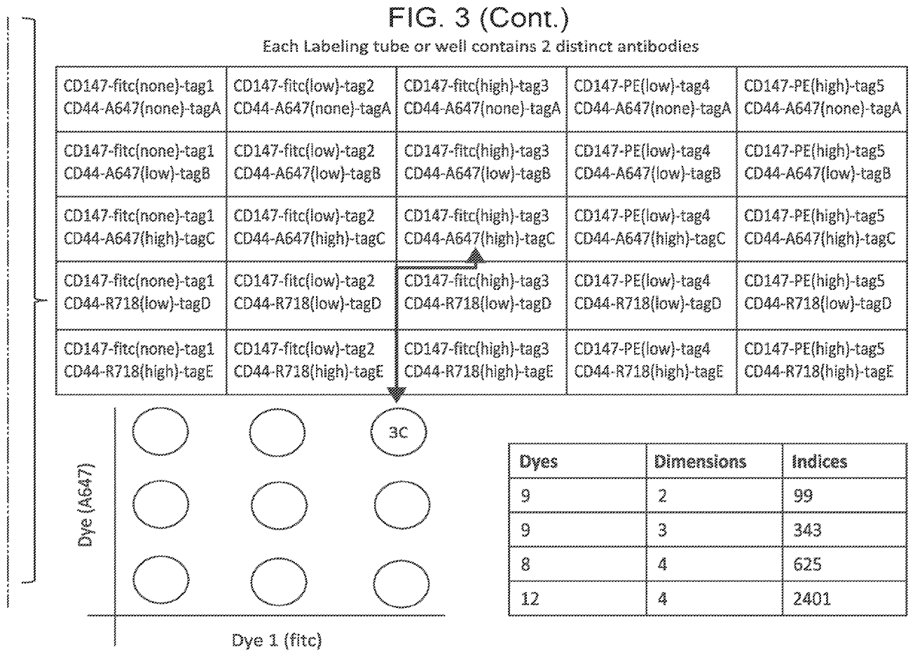

The kits may further include one or more additional components finding use in practicing embodiments of the methods. For example, the kits may include components employed producing labeled cells, e.g., macro-well plates, liquid containers, e.g., tubes, etc. In some instances, the kits may include sample indexing reagent, e.g., SMK reagents. Such reagents, where desired, may be included in any convenient format. When provided, sample indexing (e.g., SMK) reagents may be included in a multi-container format, e.g., multi-well format. For example, FIG. 3 provides an illustration of a multi-well plate that includes SMK reagents, e.g., a different combination of reagents in each well. The reagents may be present in storage stable format, e.g., dried format, such as freeze-dried format. Furthermore, the kits may include one or more components employed in obtaining sequence data, e.g., one or more of: primers, a polymerase (e.g., a thermostable polymerase, a reverse transcriptase both with hot-start properties, or the like), dsDNAse, exonuclease, dNTPs, a metal cofactor, one or more nuclease inhibitors (e.g., an RNase inhibitor and/or a DNase inhibitor), one or more molecular crowding agents (e.g., polyethylene glycol, or the like), one or more enzyme-stabilizing components (e.g., DTT), a stimulus response polymer, or any other desired kit component(s), such as devices, e.g., as described above, solid supports, containers, cartridges, e.g., tubes, beads, plates, microfluidic chips, etc. Components of the kits may be present in separate containers, or multiple components may be present in a single container.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

The following is offered by way of illustration and not by way of limitation.

### EXPERIMENTAL

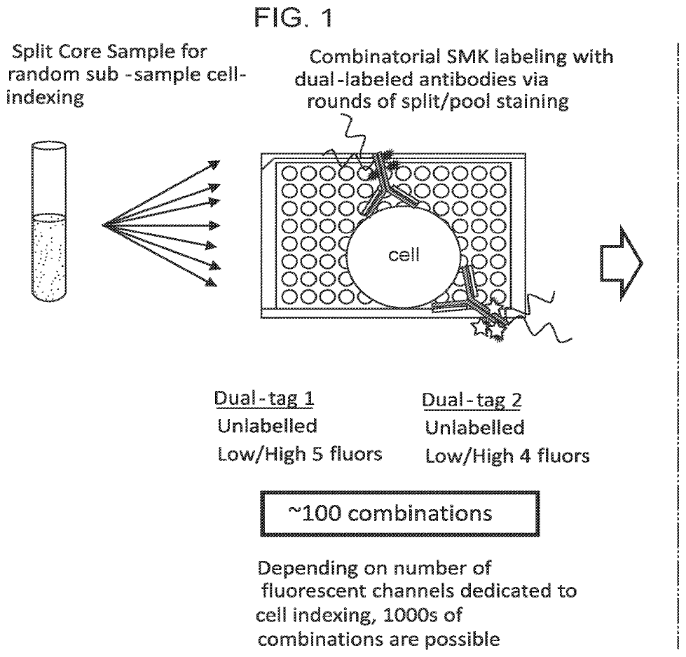

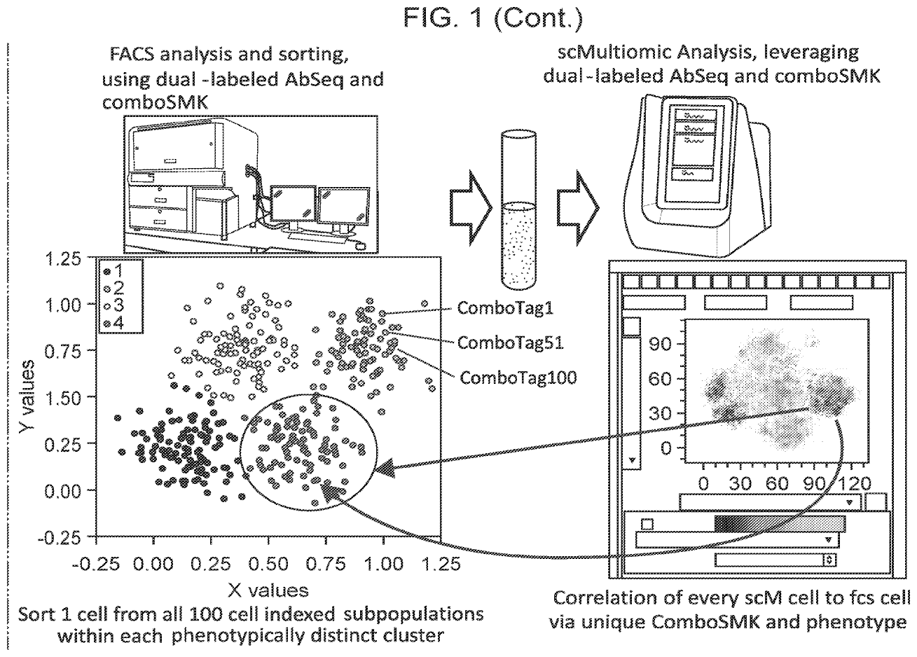

FIG. 1 provides a schematic of a workflow in accordance with an embodiment of the invention. As illustrated in FIG. 1, dual-labeled AbSeq is used in combination with Dual-labeled SMK (Single-Cell Multiplexing Kits, Becton Dickinson) tags. In this case, the combinatorial SMK labeling does not need to be as extensive (every cell does not receive a unique cell index). However, the workflow shows that one 1 unique index from each identifiable cell cluster (population) is bulk sorted via FACS. The identifiable cell populations are selected based on phenotype clustering with Ab-fluor FACS data. Because the Ab-fluors also contain an oligo barcode covalently linked, the same populations can be identified within the downstream single cell Multiomics data. Within each phenotypic cluster, the 100 cell barcodes (via combinatorial SMK) can be identified and mapped back to the corresponding cell data within the FACS data file.

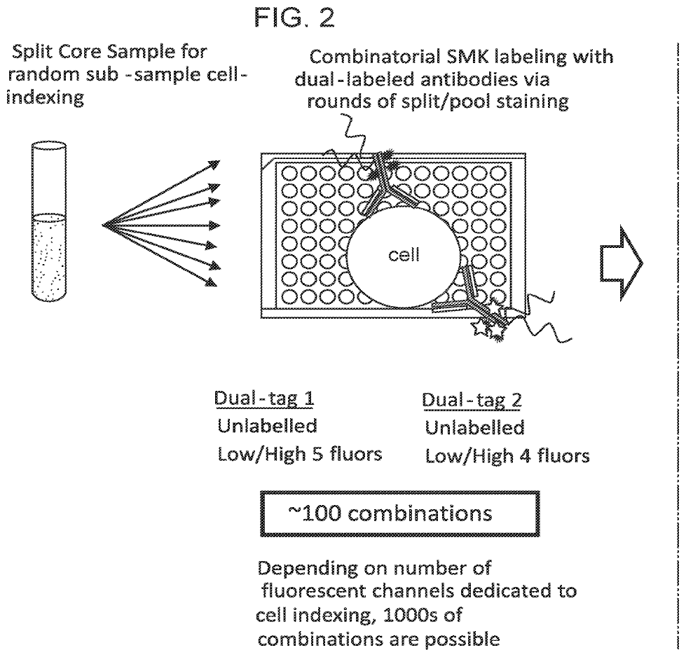

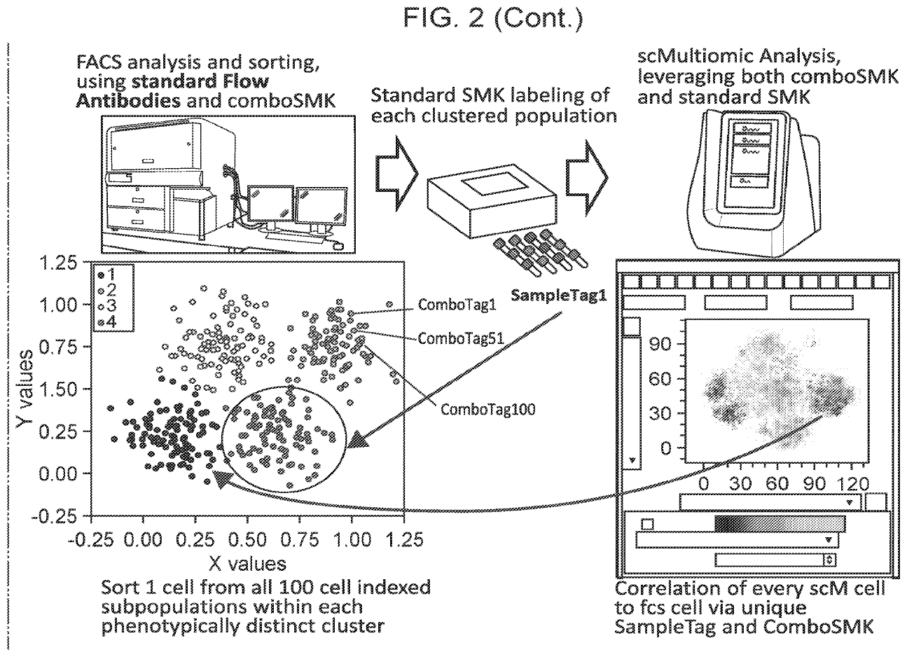

FIG. 2 provides a schematic of a workflow in accordance with an embodiment of the invention. In the workflow illustrated in FIG. 2, standard FACS ab-fluors (Becton Dickinson) are used and the workflow is not reliant on correlation of FACS population clusters to AbSeq population clusters. In this case, 100 cells from each FACS cluster of interest are sorted into individual vials. These sorted cells are then labeled with a standard cell hashing mechanism, such as BD's SMK. From there, single cell Multiomics workflow ensues (with or without AbSeq present). The standard SMK tagging provides correlation back to individual FACS populations, and from there the 100 cell indices leveraging dual-labeled combinatorial tags provide the cell by cell correlation from FACS to scM.

FIG. 3 provides details regarding combinatorial dual-labeled reagents that may be employed in embodiments of the invention and how they may be provided in a plate format.

Notwithstanding the appended claims, the disclosure is also defined by the following clauses:
1. A method of preparing an indexed population of cells from a cellular sample, the method comprising:
   splitting the cellular sample into a first plurality of portions;
   combining different portions of the first plurality with distinct first dual-indexed specific binding members having distinct fluorescence and oligonucleotide barcodes to stably associate cells of the portions with first dual-indexed specific binding members;
   combining portions of the first plurality to produce a first pool;
   splitting the first pool into a second plurality of portions; and
   combining different portions of the second plurality with distinct second dual-indexed specific binding members having distinct fluorescence and oligonucleotide barcodes to stably associate cells of the portions with second dual-indexed specific binding members;
   to produce an indexed population of cells.
2. The method according to Clause 1, wherein distinct fluorescence barcodes of the dual-indexed specific binding members comprise a unique combination of one or more fluorophores at one or more signal levels.
3. The method according to Clause 2, wherein the one or more fluorophores ranges from one to four fluorophores.
4. The method according to Clauses 2 or 3, wherein the one or more signal levels comprises from one to five signal levels.
5. The method according to any of Clauses 2 to 4, wherein the one or more fluorophores comprises a conjugated polymeric dye.
6. The method according to any of the preceding clauses, wherein distinct oligonucleotide barcodes of the dual-indexed specific binding members range in length from 10 to 500 nt.
7. The method according to any of the preceding clauses, wherein distinct oligonucleotide barcodes comprise in the 5′ to 3′ direction:
   a primer binding site;
   a dual-index bead barcode domain; and
   a capture domain.
8. The method according to Clause 7, wherein the distinct oligonucleotide barcodes have common primer binding sites and capture domains.
9. The method according to any of Clauses 7 to 8, wherein the capture domain comprises a polyA sequence.
10. The method according to any of the preceding clauses, wherein the dual-indexed specific binding members specifically bind to a cell marker.
11. The method according to Clause 10, wherein the cell marker is a surface marker or internal marker.
12. The method according Clauses 10 and 11, wherein the specific binding member specifically binds to a universal cell marker.
13. The method according to Clause 12, wherein the universal cell marker is a non-phenotype marker.
14. The method according to Clause 13, wherein the universal cell marker is selected from the group consisting of: CD44, CD45, CD47 and 3-2 micro-globulin.
15. The method according to any of the preceding clauses, wherein the specific binding member comprises an antibody or binding fragment thereof.
16. The method according to any of the preceding clauses, wherein the method further comprises combining portions of the second plurality to produce a second pool.
17. The method according to Clause 16, wherein the method further comprises:
   splitting the second pool into a third plurality of portions; and
   combining different portions of the third plurality with distinct third dual-indexed specific binding members having distinct fluorescence and oligonucleotide barcodes to stably associate cells of the portions with third dual-indexed specific binding members.
18. The method according to any of the preceding clauses, wherein the cellular sample comprises from 50 to 50,000,000 cells.
19. The method according to any of the preceding clauses, wherein the method further comprises labeling the cells with a phenotypic biomarker label.
20. The method according to Clause 19, wherein the phenotypic biomarker label comprises a fluorescently labeled specific binding member.
21. The method according to Clause 20, wherein the fluorescently labeled specific binding member comprise a fluorescently labeled antibody.
22. The method according to any of Clauses 19 to 21, wherein the phenotypic biomarker label comprises a conjugated phenotypic biomarker identifying oligonucleotide.
23. The method according to Clause 22, wherein the phenotypic biomarker label further comprises a conjugated sample identifying oligonucleotide.
24. The method according to any of the preceding clauses, wherein the method comprises combining the indexed population of cells with a second indexed population of cells produced from a second sample.
25. The method according to Clause 24, wherein the method comprises labeling cells of each combined indexed population with sample indexing oligonucleotides.
26. The method according to any of the preceding clauses, further comprising flow cytometrically assaying the indexed population of cells to obtain flow cytometric data for the indexed population of cells.
27. The method according to Clause 24, wherein the flow cytometric data comprises image data.
28. The method according to any of the preceding clauses, wherein the method further comprises obtaining sequence data for the indexed population of cells.
29. The method according to Clause 28, wherein the sequence data is obtained using a next generation sequencing protocol.
30. The method according to Clause 29, wherein the next generating sequence protocol comprises producing a sequence ready library from the indexed population of cells.
31. The method according to Clause 30, wherein the sequence ready library is produced using a barcoded bead/partition protocol.
32. The method according to any of Clauses 28 to 31, wherein the method further comprises linking sequence data with flow cytometric data for one or more of the assayed cells.
33. A population of distinct dual-indexed specific binding members each having distinct fluorescence and oligonucleotide barcodes.
34. The population according to Clause 33, wherein distinct fluorescence barcodes of the dual-indexed specific binding members comprise a unique combination of one or more fluorophores at one or more signal levels.
35. The population according to Clause 34, wherein the one or more fluorophores ranges from one to four fluorophores.
36. The population according to Clauses 34 or 35, wherein the one or more signal levels comprises from one to five signal levels.
37. The population according to any of Clauses 34 to 36, wherein the one or more fluorophores comprises a conjugated polymeric dye.
38. The population according to any of Clauses 33 to 37, wherein distinct oligonucleotide barcodes of the dual-indexed specific binding members range in length from 10 to 500 nt.
39. The population according to any of Clauses 33 to 38, wherein distinct oligonucleotide barcodes comprise in the 5′ to 3′ direction:
   a primer binding site;
   a dual-index bead barcode domain; and
   a capture domain.
40. The population according to Clause 39, wherein the distinct oligonucleotide barcodes have common primer binding sites and capture domains.
41. The population according to any of Clauses 39 to 40, wherein the capture domain comprises a polyA sequence.
42. The population according to any of Clauses 33 to 41, wherein the dual-indexed beads further comprise a cellular association member configured to provide stable association with a cell.
43. The population according to any Clauses 33 to 42, wherein the dual-indexed specific binding members specifically bind to a cell marker.
44. The population according to Clause 43, wherein the cell marker is a surface marker or internal marker.
45. The population according Clauses 43 and 44, wherein the specific binding member specifically binds to a universal cell marker.
46. The population according to Clause 45, wherein the universal cell marker is a non-phenotype marker.
47. The population according to Clause 46, wherein the universal cell marker is selected from the group consisting of: CD44, CD45, CD47 and β-2 micro-globulin.
48. The population according to any of Clauses 33 to 47, wherein the specific binding member comprises an antibody or binding fragment thereof.
49. A dual-indexed specific binding member of a population according to any of Clauses 31 to 48.
50. A kit comprising a population of dual-indexed specific binding members according to any of Clauses 31 to 48.

Although the foregoing invention has been described in some detail by way of illustration and example for purposes of clarity of understanding, it is readily apparent to those of ordinary skill in the art in light of the teachings of this invention that some changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of preparing an indexed population of cells from a cellular sample, the method comprising:
splitting the cellular sample into a first plurality of portions;
combining different portions of the first plurality with distinct first dual-indexed specific binding members having distinct fluorescence and oligonucleotide barcodes to stably associate cells of the portions with first dual-indexed specific binding members;
combining portions of the first plurality to produce a first pool;
splitting the first pool into a second plurality of portions; and
combining different portions of the second plurality with distinct second dual-indexed specific binding members having distinct fluorescence and oligonucleotide barcodes to stably associate cells of the portions with second dual-indexed specific binding members;
to produce an indexed population of cells.

2. The method according to claim 1, wherein distinct fluorescence barcodes of the dual-indexed specific binding members comprise a unique combination of one or more fluorophores at one or more signal levels.

3. The method according to claim 1, wherein distinct oligonucleotide barcodes comprise in the 5′ to 3′ direction:
a primer binding site;
a dual-index bead barcode domain; and
a capture domain.

4. The method according to claim 1, wherein the dual-indexed specific binding members specifically bind to a cell marker.

5. The method according to claim 4, wherein the cell marker is a surface marker or internal marker.

6. The method according to claim 4, wherein the specific binding member specifically binds to a universal cell marker.

7. The method according to claim 6, wherein the universal cell marker is a non-phenotype marker.

8. The method according to claim 7, wherein the universal cell marker is selected from the group consisting of: CD44, CD45, CD47 and β-2 micro-globulin.

9. The method according to claim 1, wherein the specific binding member comprises an antibody or binding fragment thereof.

10. The method according to claim 1, wherein the method further comprises labeling the cells with a phenotypic biomarker label.

11. The method according to claim 1, further comprising flow cytometrically assaying the indexed population of cells to obtain flow cytometric data for the indexed population of cells.

12. The method according to claim 11, wherein the flow cytometric data comprises image data.

13. The method according to claim 1, wherein the method further comprises obtaining sequence data for the indexed population of cells.

14. The method according to claim 11, wherein the method further comprises linking sequence data with flow cytometric data for one or more of the assayed cells.

15. A population of distinct dual-indexed specific binding members each having distinct fluorescence and oligonucleotide barcodes.

16. A dual-indexed specific binding member of a population according to claim 15.

17. A kit comprising a population of dual-indexed specific binding members according to claim 15.
